(19) United States
(12) Patent Application Publication
(10) Pub. No.: US 2019/0092792 A1
(43) Pub. Date: Mar. 28, 2019

(54) VINORELBINE MONOTARTRATE AND ITS PHARMACEUTICAL USE

(71) Applicant: SYNBIAS PHARMA AG, Schaffhausen (CH)

(72) Inventors: Oleksandr Zabudkin, Gernsbach (DE); Viktor Matviyenko, Mannheim (DE); Vladimir Matha, Borsov nad Vitavou (CZ); Christian Schickaneder, Lauf a.d. Pegnitz (DE); Iaroslav Matviienko, Mannheim (DE); Volodymyr Sypchenko, Mannheim (DE)

(73) Assignee: SYNBIAS PHARMA AG, Schaffhausen (CH)

(21) Appl. No.: 16/082,680

(22) PCT Filed: Mar. 9, 2016
(86) PCT No.: PCT/EP2016/055040

§ 371 Date: Sep. 6, 2018

(51) Int. Cl. C07D 519/04; A61P 35/00

(52) U.S. Cl. CPC C07D 519/04; A61K 9/0053; C07B 2200/13; A61P 35/00

(57) ABSTRACT

The present invention is directed to crystalline vinorelbine monotartrate and its use for the prevention and treatment of cancer, particularly non-small cell lung cancer or breast cancer. The present invention also relates to a corresponding method for the manufacture of crystalline vinorelbine monotartrate.

### FIELD OF THE INVENTION

The present invention is directed to crystalline vinorelbine monotartrate and its use for the prevention and treatment of cancer, particularly non-small cell lung cancer or breast cancer.

The present invention also relates to a corresponding method for the manufacture of crystalline vinorelbine monotartrate.

### BACKGROUND OF THE INVENTION

Vinca alkaloids, including the natural compounds vincristine and vinblastine as well as semisynthetic derivatives, such as vindesine and vinorelbine, are antimitotic drugs that are widely used in the retreatment of cancer. In general, vinca alkaloids are known to be inhibitors of mitosis and cellular proliferation. In particular, the anti-proliferative activity of the vinca alkaloid class of drugs has been shown to be due to their ability to bind tubulin.

Vinblastine and vincristine were first isolated from the leaves of Catharanthus roseus G. Don or Vinca rosea L. These alkaloids are dimers consisting of two indole units: catharanthine and vindoline. Vinblastine and vincristine first became available on the market in France in 1963 and 1964 under the brand names VELBE® and ONCOVIN®, respectively.

Vinorelbine was originally synthesized by Pierre Potier and co-workers in the 1980s. The compound is cell cycle phase-specific and interferes with the cell's ability to reproduce. Vinorelbine is commonly used in the treatment of advanced non-small cell lung cancer (single agent or as part of a combination therapy) and of metastatic breast cancer after failure of standard first line chemotherapy or after relapse within 6 months of anthracycline based adjuvant therapy and aggressive fibromatosis.

In all known pharmaceutical formulations, vinorelbine is used in form of a bitartrate salt. Vinorelbine bitartrate is a white to yellow or light brown amorphous powder that is particularly unstable in solid form being sensitive to both humidity and light. Hence, it has to be kept in tightly closed, light-resistant containers and stored in a freezer below −15° C. However, solutions of vinorelbine bitartrate can be kept at temperatures between 3-5° C. This is the case for both water-based solutions for injectable preparations, and for soft capsules filling solutions composed of liquid polyethylene glycol, glycerol, ethanol, and water.

An injectable formulation of vinorelbine was launched in France in 1989 under the brand name Navelbine®. However, in order to avoid problems associated with intravenous drug delivery route, there was a continued need for an oral vinorelbine dosage form with similar efficacy as the intravenous formulation. However, it has turned out to be difficult to develop such oral dosage form, primarily due to the instability of vinorelbine.

International patent publication WO 2003/101383 A2 describes the first oral formulation available on the market, a soft gelatin capsule containing vinorelbine bitartrate dissolved in an excipient mixture comprising polyethylene glycol, glycerol, ethanol, and water. This formulation is known under the brand name Navelbine Oral®. Although commercially successful, the soft gelatin capsules filled with a liquid vinorelbine composition provides for a rather challenging and costly technology requiring the active ingredient to be continuously maintained in solution inside the capsule. This capsules have low stability under ambient conditions and have to be stored in the refrigerator at 5° C. Furthermore, after long-term storage at this temperature the total amount of impurities has been shown to be significantly increased.

Another approach how to stabilize vinorelbine bitartrate was its dispersion in a mixture with polyethylene glycol, preferably in a mass ratio of 1:3 to 1:6, as described in International patent publication WO 2006/069938 A1. The dispersion can be distributed in a hard gelatin capsule, as divided pellets or associated with compression excipients in form of a tablet. But again, the task of this formulation can be seen in the amount of impurities after long-term storage, thus resulting in complex logistics with respect to continuous supply with vinorelbine.

International patent publication WO 2009/007389 A1 describes a solid dosage form made from conventional excipients and a water-soluble vinorelbine salt in order to facilitate manufacture. Manufacturing methods may include wet granulation or dry mixing of different components followed filling them into hard gelatin capsules or by compressing them into film-coated tablets. The oral dosage form according to WO 2009/007389 A1 comprises, in addition to the vinorelbine salt, at least one diluent and at least one lubricant. However, these solid dosage forms still have low stability under normal conditions and are stable only at 5° C. for a period of 12 months.

Despite these achievements with respect to pharmaceutical formulation, however, the clinical applicability of vinorelbine bitartrate as active pharmaceutical ingredient for the production of stable oral dosage form still remains hampered due to persistent problems with the stability, solubility and/or bioavailability of the compound. Thus, there is still an ongoing need for alternative to vinorelbine bitartrate, that is, a water-soluble vinorelbine salt that is stable in solid form, and thus can be directly formulated in a pharmaceutical composition in a comparably simple and cost-efficient manner without compromising for solubility and/or bioavailability of the active ingredient.

It is therefore an objective of the present invention to provide pharmaceutically acceptable forms of vinorelbine monotartrate having a good chemical and/or physical stability and/or good processability, both during its preparation as an active pharmaceutical ingredient as well as in the preparation of pharmaceutical compositions containing vinorelbine.

It was found that crystalline vinorelbine monotartrate forms as described below may provide beneficial properties especially regarding stability issues and may furthermore enhance the performance of oral dosage forms comprising said vinorelbine monotartrate crystalline forms.

In addition, combined with suitable excipients, the crystalline vinorelbine may provide a good means for development of oral pharmaceutical formulation as well as the process.

Accordingly, it is an object of the present invention to provide for a stable vinorelbine salt that overcomes the above limitations as well as a corresponding method for its production.

### SUMMARY OF THE INVENTION

The invention relates to crystalline vinorelbine monotartrate forms including different solvates and hydrate forms, processes for the preparation thereof, as well as pharmaceutical compositions and formulations comprising said crystalline forms.

In another aspect, the present invention relates to stable crystalline forms of vinorelbine, comprised as active ingredient in pharmaceutical compositions, preferably for oral administration, wherein the crystalline forms of vinorelbine is a monotartrate represented as solvates or a hydrate.

The pharmaceutical composition of the present invention may comprise a mixture of one or more excipients.

In yet another aspect, the pharmaceutical composition consists of crystalline vinorelbine monotartrate and one excipient, and in particularly consists of crystalline vinorelbine monotartrate and one co-processed excipient.

In a further aspect, the present invention relates to the pharmaceutical composition as defined herein for use in the prevention and/or treatment of cancer, in particular non-small cell lung cancer and breast cancer.

Further embodiments of the present invention become apparent from the following detailed description and the appended drawings.

### BRIEF DESCRIPTION OF THE DRAWINGS

FIG. 1 depicts the results of X-ray powder diffraction analysis of crystalline vinorelbine monotartrate acetone solvate.

FIG. 2 depicts the results of X-ray powder diffraction analysis of crystalline vinorelbine monotartrate diethyl ketone solvate.

FIG. 3 depicts the results of X-ray powder diffraction analysis of crystalline vinorelbine monotartrate ethyl acetate solvate.

FIG. 4 depicts the results of X-ray powder diffraction analysis of crystalline vinorelbine monotartrate isopropanol solvate.

FIG. 5 depicts the results of X-ray powder diffraction analysis of crystalline vinorelbine monotartrate hydrate.

FIG. 6 depicts the results of X-ray powder diffraction analysis of amorphous vinorelbine monotartrate.

FIG. 7 depicts representative thermogravimetric analysis (TGA) for crystalline vinorelbine monotartrate hydrate according to the present invention.

FIG. 8 depicts representative differential scanning calorimetry (DSC) analysis (closed CSC cells) of crystalline vinorelbine monotartrate hydrate according to the present invention.

FIG. 9 depicts the results of dissolution test of HG capsules comprising crystalline vinorelbine monotartrate acetone solvate in three different dissolution media.

FIG. 10 depicts the results of dissolution test of HG capsules comprising crystalline vinorelbine monotartrate hydrate in three different dissolution media.

FIG. 11 depicts the results of a representative X-ray powder diffraction analysis for the mixture of crystalline vinorelbine monotartrate hydrate and excipient filling in capsules according to the present invention.

### DETAILED DESCRIPTION OF THE INVENTION

The present invention is based on the unexpected finding that vinorelbine monotartrate can be readily provided in crystalline form and that such crystalline vinorelbine monotartrate represents a superior active ingredient (as compared to the commonly used vinorelbine bitartrate) for the treatment of cancer, particularly non-small cell lung cancer or breast cancer, which exhibits pronounced thermo- and photostability without compromising for solubility and/or bioavailability, thus facilitating long-term storage. Furthermore, it has been found that crystalline vinorelbine monotartrate can be directly processed and formulated in a pharmaceutical composition, which results in a simple and cost-effective manufacturing process for providing a vinorelbine containing medicament, preferably an oral dosage form.

The present invention will be described in the following with respect to particular embodiments and with reference to certain drawings but the invention is to be understood as not limited thereto but only by the appended claims. The drawings described are only schematic and representative and are to be considered non-limiting.

Where the term “comprising” is used in the present description and claims, it does not exclude other elements or steps. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Where an indefinite or definite article is used when referring to a singular noun e.g. “a”, “an” or “the”, this includes a plural of that noun unless specifically stated otherwise.

In case, numerical values are indicated in the context of the present invention the skilled person will understand that the technical effect of the feature in question is ensured within an interval of accuracy, which typically encompasses a deviation of the numerical value given of +10%, and preferably of 5%.

Furthermore, the terms first, second, third, (a), (b), (c), and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Further definitions of term will be given in the following in the context of which the terms are used. The following terms or definitions are provided solely to aid in the understanding of the invention. These definitions should not be construed to have a scope less than understood by a person of ordinary skill in the art.

The term “solvate”, as used herein and unless indicated otherwise, refers to a crystal form that incorporates a solvent in the crystal structure. When the solvent is water, the solvate is referred to as a “hydrate”. The solvent in a solvate may be present in either a stoichiometric or in a non-stoichiometric amount.

Typically, the crystalline vinorelbine monotartrate solvate comprises less than 25% (w/w) or less than 20% (w/w) residual solvents included in the crystal structure (i.e., weight of total residual solvents based on the total weight of the crystalline form), that is, solvent molecules being integrated in or associated to the crystal structure.

In particular embodiments, the crystalline vinorelbine monotartrate solvate comprises less than 15% (w/w) or less than 13% (w/w) or less than 11% (w/w) residual solvents, such as 14.5%, 14.0%, 13.5%, 13.0%, 12.5%, 12.0%, 11.5%, 11.0%, 10.5% or 10.0% (w/w each). In preferred embodiments, the crystalline vinorelbine monotartrate solvate comprises less than 10% (w/w) or less than 7% (w/w) or less than 3% (w/w) residual solvents, such as 9.5%, 9.0%, 8.5%, 8.0%, 7.5%, 7.0%, 6.5%, 6.0%, 5.5%, 5.0%, 4.5%, 4.0%, 3.5%, 3.0%, 2.5%, 2.0%, 1.5%, 1.0%, or 0.5% (w/w each). The solvent is typically water or an organic solvent, such as an alcohol, ester, an ether or a ketone or a mixture thereof. In preferred embodiments, the organic solvent is selected from the group consisting of acetone, diethyl ketone, ethyl acetate or isopropanol or a mixture thereof.

In specific embodiments, the vinorelbine monotartrate solvate comprises a molar ratio of vinorelbine monotartrate to solvent in the range from 4:1 to 1:6 or in the range from 2:1 to 1:5, and preferably in the range from 1:1 to 1:3.

The term “crystalline”, as used herein, is to be understood in the common sense, that is, that the vinorelbine monotartrate is present in crystalline (i.e. non-amorphous) form being obtained for example by crystallization of the compound from a solvent.

The term “photostability”, as used herein, is to be understood such that a sample of the product to be analyzed in a quantity of about 14 mg is placed in a 10 ml light glass volumetric flask and is exposed in a photo chamber to a xenon lamp (wave length 300-800 nm; fluence rate 250-765 W/m2). The amount of the known photo-degradation product 3,6-epoxy vinorelbine is determined after a specific amount of time of exposure by means of HPLC (High-performance liquid chromatography). The described photostability test procedure corresponds to the photostability determining method described in European Pharmacopoeia 7.0 using exposure time 2 hours.

The term “thermostability”, as used herein, is to be understood such that a sample of the product to be analyzed is incubated at a certain temperature. The degradation impurities are determined after a specific amount of time of incubation by means of HPLC.

The term “degradation impurities”, as used herein, is to be understood such a sample of the relevant product is subjected to HPLC analysis and calculation of the content of impurities in Vinorelbine is performed according to the method described in European Pharmacopoeia 7.0.

Only one method for the preparation of vinorelbine monotartrate in a solid state has been reported in the art (CN1437942A), and that is by precipitation with diethyl ether from acetone solution containing vinorelbine monotartrate. The reproduction of process described in CN 1437942A results in an amorphous solid. The addition of different anti-solvents to a solution of vinorelbine monotartrate in an organic solvent also leads to the formation of an amorphous solid.

The amorphous vinorelbine monotartrate has been found to be unstable upon exposure to elevated temperature and humidity for an extended period, therefore amorphous vinorelbine monotartrate cannot be used for the preparation of the stable oral dosage formulations.

It has been unexpectedly found that in contrast to vinorelbine bitartrate, vinorelbine monotartrate can form a variety of crystal forms. Such crystal forms include organic solvate and hydrate forms. Crystalline forms of vinorelbine monotartrate can be used as an excellent active ingredient (either in form of organic solvates or in form of a hydrate) for the manufacture of a pharmaceutical composition with pronounced thermo- and photostability without affecting solubility and/or bioavailability of the active ingredient.

Crystalline vinorelbine monotartrate organic solvates (e.g. acetone solvate, diethyl ketone solvate, isopropanol solvate, ethyl acetate solvate) may be prepared by a process comprising:
(a) providing a solution of vinorelbine monotartrate in a liquid containing at least one organic solvent (for example in methylene chloride);
(b) drying the solution of vinorelbine monotartrate in a liquid containing at least one organic solvent until a dry residue is obtained;
(c) dissolving the dry residue in a liquid containing at least one organic solvent to obtain a mixture;
(d) maintaining the mixture under heating and stirring to obtain a solid precipitate;
(e) isolating the solid precipitate;
(f) drying the solid precipitate.

The obtained crystalline solvates contain organic solvent in either a stoichiometric or in a non-stoichiometric amount and do not desolvate during drying under vacuum at 60° C.

Crystalline vinorelbine monotartrate solvates are characterized by a powder X-ray diffraction pattern comprising peaks at average diffraction angles (20). The XRD patterns of some crystalline vinorelbine monotartrate solvates are presented in the Table 1.

TABLE 1
XRD patterns of some crystalline vinorelbine
monotartrate solvates

| Type of | Significant peaks at average diffraction angles | | | | | | | | | |
|---|---|---|---|---|---|---|---|---|---|---|
| solvate | (2Θ) | | | | | | | | | |
| Acetone | 7.9 | 9.5 | 10.1 | 13.2 | 13.4 | 14.4 | 16.7 | 16.9 | 19.1 | 20.7 |
| solvate | | | | | | | | | | |
| Diethyl | 5.4 | 9.5 | 10.1 | 10.4 | 10.8 | 12.9 | 13.1 | 14.8 | 16.2 | 20.2 |
| ketone | | | | | | | | | | |
| solvate | | | | | | | | | | |
| Ethyl | 9.4 | 9.9 | 10.0 | 10.7 | 10.9 | 13.0 | 14.1 | 15.6 | 16.2 | 20.2 |
| acetate | | | | | | | | | | |
| solvate | | | | | | | | | | |
| Iso- | 8.0 | 9.2 | 10.3 | 10.8 | 10.9 | 13.4 | 14.6 | 16.9 | 19.3 | 22.9 |
| propanol | | | | | | | | | | |
| solvate | | | | | | | | | | |

It has been surprisingly found that the obtained crystalline vinorelbine monotartrate solvates have a high stability.

In further preferred embodiments, the crystalline vinorelbine monotartrate solvate according to the present invention is characterized by its stability, that is, the compound can be stored under typical storage conditions for at least three months or for at least six months, and without appreciable degradation (in particular, thermodegradation and/or photodegradation). In specific embodiments, the compound can be stored for at least 24 months without appreciable degradation.

In particular embodiments, the crystalline vinorelbine monotartrate acetone solvate according to the present invention is characterized by a thermostability of less than 0.10% degradation after 6 months at 5° C.±3° C.).

The term “degradation”, as used herein, is to be understood to relate to the total amount of identified (e.g. 3,6-epoxy vinorelbine, 4-O-deacetylvinorelbine and vinorelbine N-oxide) and unidentified degradation products that form in a sample after a particular incubation period.

Typically, degradation after 3 months at 5° C.±3° C. is less than 0.02% or less than 0.01%. Preferably, degradation after 6 months at 5° C.±3° C. is less than 0.10% or less than 0.05%.

In preferred embodiments, the crystalline vinorelbine monotartrate acetone solvate according to the present invention is further characterized by a thermostability of less than 0.10% degradation after 6 month at 25° C.±2° C. Particularly preferably, the crystalline vinorelbine monotartrate is further characterized by a thermostability of less than 0.20% degradation after 2 months at 40° C.±2° C.

Particularly preferably, the crystalline vinorelbine monotartrate according to the present invention is further characterized by a thermostability of less than 0.10% or less than 0.05% degradation after 6 months at 25° C.±2° C. Typically, degradation after 6 months at 25° C.±2° C. is less than 0.05% or less than 0.02%.

In further preferred embodiments, the crystalline vinorelbine monotartrate according to the present invention is further characterized by a thermostability of less than 0.20% or less than 0.15% degradation after 2 months at 40° C.±2° C.

Various types of vinorelbine monotartrate solvates remain stable even under stressing conditions at 60° C. After one week storage of acetone solvate and isopropanol solvate at 60° C. they didn't show any chemical degradation in contrast to the vinorelbine bitartrate.

However, organic solvates are rarely used in pharmaceuticals because the number of pharmaceutically acceptable solvents is very small and the solvents are volatile thus making it difficult to maintain the solvent in the crystal.

It was surprisingly found that a crystalline vinorelbine monotartrate hydrate can be obtained from any of the vinorelbine monotartrate solvates by exposing solvate forms to air with different relative humidity levels and temperatures. Air or an inert gas with different relative humidity levels and temperatures is also referred to as “water vapour”.

The present invention provides a process for the preparation of vinorelbine monotartrate hydrate by exposing solvate forms to water vapour. The hydrate forms prepared from different solvates have the same crystal structure and the content of the residual solvents was found to be within ICH limits.

Conversion of solvates into hydrate is performed in different controlled conditions. The temperature during conversion is in an interval from 20 to 70° C., preferable from 40 to 60° C., relative humidity is from 30% RH to 75% RH, preferably from 40 to 60% RH. The conversion time is between 8 and 48 hours, preferably between 16 and 32 hours.

Vinorelbine monotartrate hydrate is obtained as a result of a substitution of organic solvents with water. The water content after such substitution is between 0.5 and 10 w/w %, preferable, between 3 and 7 w/w %.

The crystalline vinorelbine monotartrate hydrate according to the present invention is characterized by a x-ray powder diffraction pattern comprising significant peaks at average diffraction angles (2Θ) of 7.9°, 9.5°, 10.3°, 10.8°, and 13.4°, 13.6°, 14.5° and (each ±0.2°).

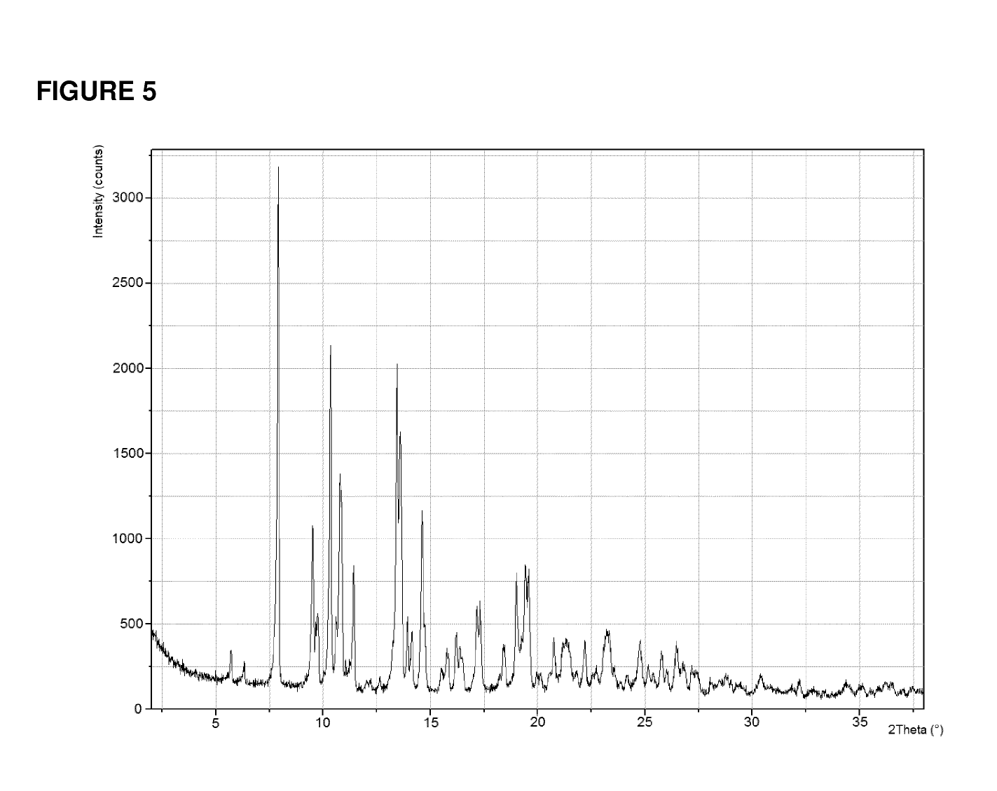

In a specific embodiment, the crystalline vinorelbine monotartrate hydrate is characterized by a powder X-ray diffraction pattern as illustrated in Table 2 and FIG. 5.

TABLE 2
PXRD peak table for crystalline
vinorelbine monotartrate hydrate

| Pos. | Height | FWHM | d-spacing | Rel. Int. |
|---|---|---|---|---|
| [°2θ] | [cts] | Left [°2θ] | [Å] | [%] |
| 5.6603 | 155.39 | 0.0640 | 15.61370 | 9.70 |
| 6.3047 | 64.91 | 0.0640 | 14.01933 | 4.05 |
| 7.8920 | 1601.52 | 0.0640 | 11.20280 | 100.00 |
| 9.5022 | 619.80 | 0.0768 | 9.30778 | 38.70 |
| 9.6400 | 526.70 | 0.0512 | 9.17505 | 32.89 |
| 10.3129 | 1349.49 | 0.1151 | 8.57780 | 84.26 |
| 10.7438 | 1179.93 | 0.0895 | 8.23476 | 73.68 |
| 10.8241 | 803.11 | 0.0640 | 8.17386 | 50.15 |
| 11.0432 | 149.93 | 0.0640 | 8.01218 | 9.36 |
| 11.4028 | 398.03 | 0.0895 | 7.76029 | 24.85 |
| 12.0440 | 46.35 | 0.0768 | 7.34854 | 2.89 |
| 13.4144 | 1450.23 | 0.0640 | 6.60074 | 90.55 |
| 13.5607 | 1344.16 | 0.0895 | 6.52987 | 83.93 |
| 13.9826 | 253.57 | 0.1279 | 6.33378 | 15.83 |
| 14.1259 | 211.20 | 0.0512 | 6.26981 | 13.19 |
| 14.5737 | 764.07 | 0.1151 | 6.07818 | 47.71 |
| 15.4909 | 82.37 | 0.0640 | 5.72031 | 5.14 |
| 15.8024 | 114.03 | 0.1023 | 5.60824 | 7.12 |
| 16.2052 | 242.01 | 0.0895 | 5.46972 | 15.11 |
| 16.5319 | 99.57 | 0.0512 | 5.36236 | 6.22 |
| 17.1502 | 389.84 | 0.0640 | 5.17042 | 24.34 |
| 17.2971 | 364.86 | 0.0640 | 5.12684 | 22.78 |
| 18.3869 | 191.25 | 0.1535 | 4.82534 | 11.94 |
| 18.9855 | 427.01 | 0.0768 | 4.67454 | 26.66 |
| 19.4119 | 655.89 | 0.0640 | 4.57280 | 40.95 |
| 19.5577 | 490.86 | 0.0512 | 4.53904 | 30.65 |
| 20.0486 | 75.58 | 0.2558 | 4.42900 | 4.72 |
| 20.7169 | 244.33 | 0.0640 | 4.28761 | 15.26 |
| 21.1602 | 278.41 | 0.1023 | 4.19877 | 17.38 |
| 21.4709 | 224.42 | 0.0768 | 4.13871 | 14.01 |
| 22.2069 | 220.45 | 0.0640 | 4.00318 | 13.76 |
| 22.7214 | 119.84 | 0.1023 | 3.91369 | 7.48 |
| 23.1938 | 350.58 | 0.0512 | 3.83504 | 21.89 |
| 23.5706 | 93.54 | 0.1279 | 3.77457 | 5.84 |
| 24.1472 | 87.34 | 0.0768 | 3.68573 | 5.45 |
| 24.7625 | 234.71 | 0.1535 | 3.59552 | 14.66 |
| 25.1137 | 105.37 | 0.1535 | 3.54604 | 6.58 |
| 25.7867 | 191.69 | 0.0895 | 3.45499 | 11.97 |
| 26.4846 | 207.94 | 0.1535 | 3.36551 | 12.98 |
| 26.8187 | 116.81 | 0.1279 | 3.32434 | 7.29 |
| 27.4412 | 100.78 | 0.3582 | 3.25033 | 6.29 |
| 28.2760 | 47.50 | 0.3070 | 3.15624 | 2.97 |
| 28.7915 | 81.31 | 0.2558 | 3.10089 | 5.08 |
| 29.4985 | 48.48 | 0.1535 | 3.02816 | 3.03 |
| 30.3843 | 100.92 | 0.0768 | 2.94186 | 6.30 |
| 30.8031 | 46.71 | 0.1535 | 2.90282 | 2.92 |
| 32.2225 | 32.43 | 0.1535 | 2.77812 | 2.03 |
| 32.8210 | 16.73 | 0.2047 | 2.72881 | 1.04 |
| 33.3521 | 10.14 | 0.1535 | 2.68656 | 0.63 |
| 34.3526 | 60.81 | 0.1791 | 2.61058 | 3.80 |
| 35.1208 | 29.33 | 0.2558 | 2.55522 | 1.83 |
| 36.1536 | 42.01 | 0.1535 | 2.48456 | 2.62 |

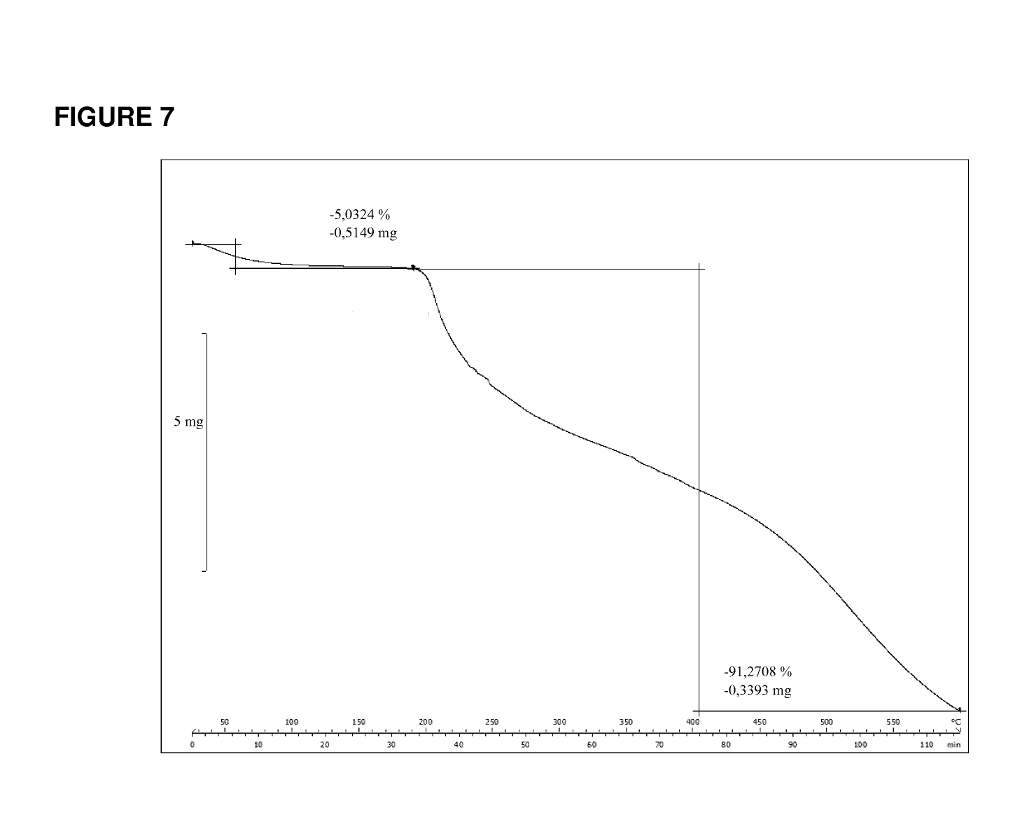

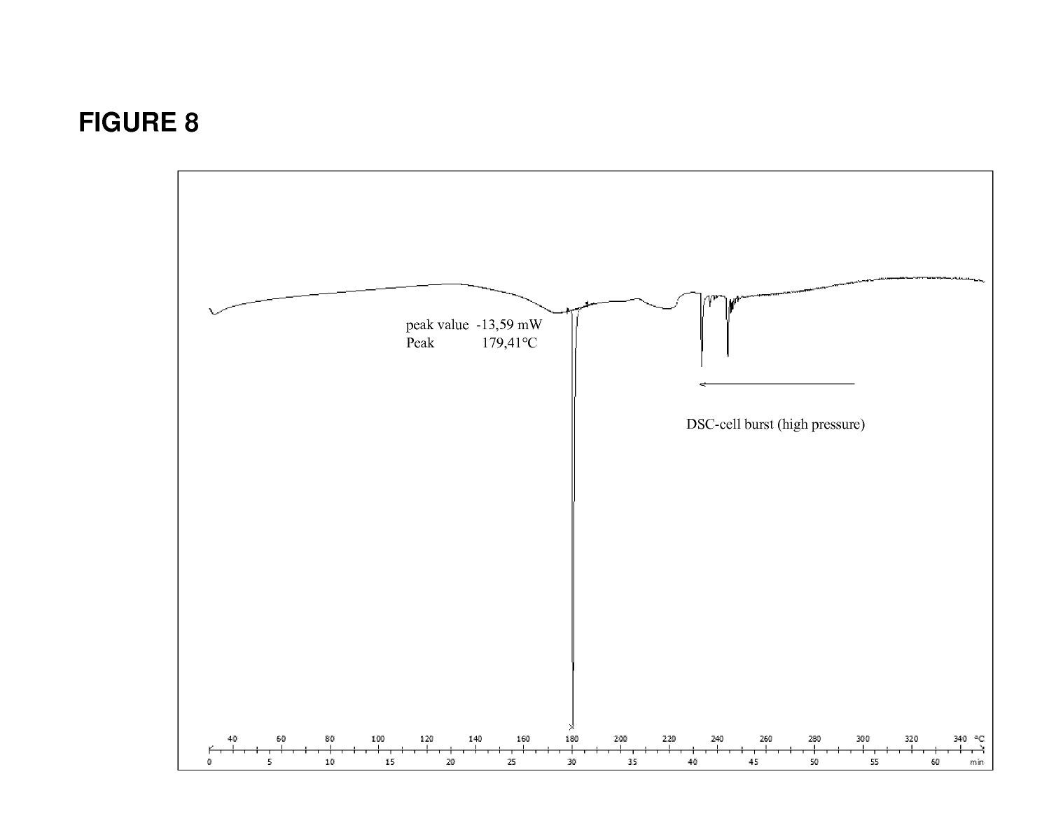

The crystalline vinorelbine monotartrate hydrate of the present invention is also characterized by its differential scanning calorimetry (DSC) thermogram as depicted in FIG. 8, with sharp endothermic signal at 179.4° C. The crystalline vinorelbine monotartrate hydrate of the present invention is characterized by a Thermogravimetric Analysis (TGA) thermogram as depicted in FIG. 7.

Stability studies supported surprisingly a very high stability of crystalline vinorelbine monotartrate hydrate.

The crystalline vinorelbine monotartrate hydrate according to the present invention is characterized by its stability, that is, the compound can be stored under typical storage conditions for at least three months or for at least six months, and without appreciable degradation (in particular, thermodegradation and/or photodegradation). In specific embodiments, the compound can be stored for at least 24 months without appreciable degradation.

In particular embodiments, the crystalline vinorelbine monotartrate hydrate according to the present invention is characterized by a thermostability of less than 0.05% degradation after 6 months at 25° C.).

The term “degradation”, as used herein, is to be understood to relate to the total amount of identified (e.g. 3,6-epoxy vinorelbine, 4-O-deacetylvinorelbine and vinorelbine N-oxide) and unidentified degradation products that form in a sample after a particular incubation period.

Typically, degradation after 3 months at 25° C. is less than 0.10% or less than 0.05%. Preferably, degradation after 6 months at 25° C. is less than 0.05% or less than 0.02%.

In preferred embodiments, the crystalline vinorelbine monotartrate is further characterized by a thermostability of less than 0.15% degradation after 2 months at 40° C.±2° C.

Various types of vinorelbine monotartrate solvates as well as hydrate remain stable even under stressing conditions at 60° C. After one week storage at 60° C. vinorelbine monotartrate hydrate is characterized by a thermostability of less than 0.10% degradation.

In further particular embodiments, the crystalline vinorelbine monotartrate solvate or hydrate according to the present invention is further characterized by a photostability (as determined by the amount of 3,6-epoxy vinorelbine produced) of less than 0.3% or less than 0.2% degradation after 30 min of illumination of samples or less than 0.7% or less than 0.5% degradation after 120 min of illumination of samples in contrast with vinorelbine bitartrate as shown in Table 11.

In a further aspect, the present invention relates to the crystalline vinorelbine monotartrate of the present invention for use in the prevention and/or treatment of cancer. In preferred embodiments, the crystalline vinorelbine monotartrate of the present invention is for use in the prevention and/or treatment of non-small cell lung cancer or breast cancer.

In yet another aspect, the present invention relates to a pharmaceutical composition comprising the crystalline vinorelbine monotartrate of the present invention.

The pharmaceutical composition can be formulated employing conventional solid or liquid vehicles or diluents and pharmaceutical additives of a type appropriate to the mode of desired administration.

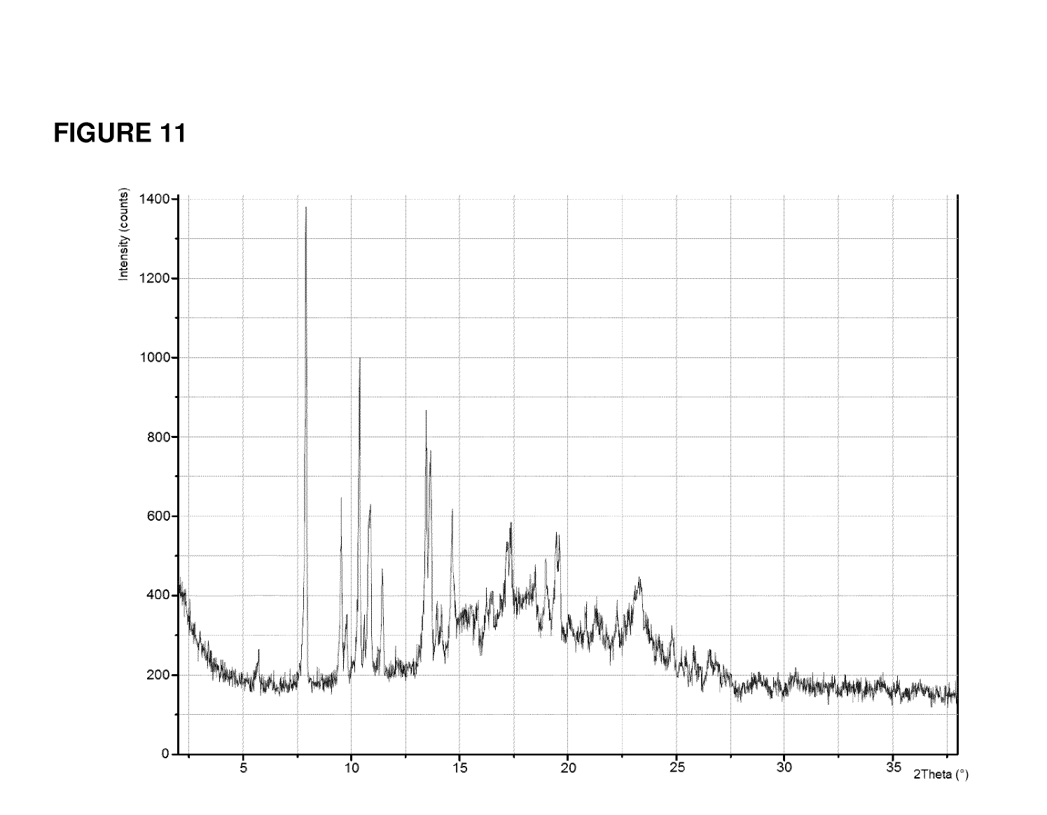

Due to the crystalline nature of the vinorelbine monotartrate the pharmaceutical composition is typically a solid composition, with the active pharmaceutical ingredient vinorelbine monotartrate being provided in crystalline form (FIG. 11). The pharmaceutical composition may be administered via any route of administration, local or systemic, such as parenteral, topical, and oral, with oral administration being particularly preferred. In other preferred embodiments, the pharmaceutical composition is provided as dosage form, that is, as a ready-to-use formulation.

Particularly preferably, the pharmaceutical composition in accordance with the present invention is a solid oral dosage form, that is, a formulation that is ready-to-use for oral administration. In preferred embodiments, the solid oral dosage form is selected from the group consisting of capsules, tablets, pills, granules, pellets, and powder, with capsules and tablets being most preferred. In highly preferred embodiments, the capsules are gelatin hydroxypropylmethyl cellulose or pullan, capsules, with hard gelatin capsules being particularly preferred. In other highly preferred embodiments, the tablets are obtained by direct compression or dry compaction.

Both capsules and tablets may be uncoated or coated including a tablet core and an inner seal coating layer coated on the tablet core.

All these oral dosage forms are well established in the art (see, e.g., Gennaro, A. L. and Gennaro, A. R. (2000), Remington: The Science and Practice of Pharmacy, 20th Ed., Lippincott Williams & Wilkins, Philadelphia, Pa.; Crowder, T. M. et al. (2003) A Guide to Pharmaceutical Particulate Science. Interpharm/CRC, Boca Raton, Fla.; Niazi, S. K. (2004) Handbook of Pharmaceutical Manufacturing Formulations, CRC Press, Boca Raton, Fla.; Podczeck, F. and Jones, B. E. (2004) Pharmaceutical Capsules, 2nd Ed., Pharmaceutical Press, London).

The amount of crystalline vinorelbine monotartrate present in the pharmaceutical composition typically corresponds to an equivalent of 5-250 mg vinorelbine base or of 10-200 mg vinorelbine base, and preferably to an equivalent of 15-150 mg vinorelbine base. In particular embodiments, the amount of active ingredient present in the pharmaceutical composition corresponds to an equivalent of 20-100 mg vinorelbine base, such as an amount corresponding to an equivalent of 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg vinorelbine base. The molecular weight of vinorelbine base is 778.93, whereas the molecular weight of vinorelbine monotartrate is 929.03.

According to the present invention, it is to be understood that the active ingredient is present in the pharmaceutical composition in any amount being effective to achieve the desired pharmacological effect such as to stop tumor progression or to induce an apoptotic effect in tumor cells when administered to a patient. Effective amounts are generally chosen in accordance with a number of factors, e.g., the age, size and general condition of the patient and the medical condition being treated, and determined by a variety of means, for example, dose ranging trials, well known to, and readily practiced by persons of ordinary skill in art. The daily dosage of crystalline vinorelbine monotartrate to be administered to a subject typically corresponds to an equivalent of 5-1000 mg vinorelbine base or of 10-500 mg vinorelbine base or of 10-200 mg vinorelbine base, and preferably to an equivalent of 20-100 mg vinorelbine base.

In yet other preferred embodiments, the pharmaceutical composition of the present invention, such as a solid oral dosage form, is characterized by a thermostability of less than 0.15% or less than 0.10% or less than 0.05% degradation after 2 months at 25° C.±2° C. Particularly, degradation after six months is less than 0.2% or less than 0.15% or less than 0.10%, and particularly preferably degradation after six months is less than 0.15% or less than 0.10%.

In further particularly preferred embodiments, the pharmaceutical composition of the present invention, such as a solid oral dosage form, is characterized by a thermostability of less than 0.2% or less than 0.15% or less than 0.10% degradation after 3 months at 40° C.±2° C.

A pharmaceutical composition of the present invention may comprise at least one excipient, particularly at least one co-processed excipient. Typically, the pharmaceutical composition of the present invention comprises a single excipient but may also comprise a mixture of two or more excipients, for example in form of a co-processed excipient. In preferred embodiments, the pharmaceutical composition is devoid of polyethylene glycol.

The term “excipient”, as used herein, refers to any substances, other than the active ingredients, in a pharmaceutical composition, which have been appropriately evaluated for safety and are included in a drug delivery system to either aid the processing or to aid manufacture, protect, support, enhance stability, bioavailability or patient acceptability, assist in product identification, or enhance any other attributes of the overall safety and effectiveness of the drug delivery system during storage or use.

The term “co-processed excipient”, as used herein, can be defined as combining two or more established excipients. Co-processing of excipients could lead to the formation of excipients with superior properties compared to the simple physical mixtures of their components, for example, with respect to better flowability, improved compressibility, better dilution potential, reworkability, stability, fewer fill weight variation, and controlled particle size. The aim of co-processing is to obtain a product with added value related to the ratio of its functionality/price.

The development of co-processed excipients starts with the selection of the excipients to be combined, their targeted proportion, selection of preparation method to get optimized product with desired physico-chemical parameters and it ends with minimizing avoidance with batch-to-batch variations. An excipient of reasonable price has to be combined with the optimal amount of a functional material in order to obtain integrated product, with superior functionality than the simple mixture of components. Co-processing is interesting because the products are physically modified in a special way without altering the chemical structure. A fixed and homogenous distribution for the components is achieved by embedding them within mini-granules. Segregation is diminished by adhesion of the actives on the porous particles making process validation and in process control easy and reliable (reviewed inter alia in Gohel, M. C. and Jogani, P. D. (2005) J. Pharm. Pharmaceut. Sci. 8, 76-93).

Commercially available examples of “co-processed excipients” to be employed in the pharmaceutical composition of the present invention include inter alia fructose/starch (Advantose FS-95; SPI Polyols, France), microcrystalline cellulose/guar gum (Avicel CE-15; FMC, USA), microcrystalline cellulose/lactose (Cellactose; Meggle, Germany), sucrose/dextrin (DI-PAC; American Sugar, USA), lactose/PVP/crospovidone (Ludipress; BASF, Ludwigshafen), granulated mannitol (Pearlitol SD; Roquette, France), anhydrous lactose/lactitol (Pharmatose DCL40; DMV, Netherlands), vinyl acetate/vinyl pyrollidone (Plasdone S-630; ISP, USA), microcrystalline cellulose/colloidal silica (Prosolv, Pen West, USA), and lactose/maize starch (Starlac; Roqette, France). The respective brand names and exemplary manufacturers are given in examples of “co-processed excipient” being specifically adapted to the production of oral dosage forms include inter alia mannitol/cellulose (for example, 50:50 (w/w); 60:40 (w/w), or 70:30 (w/w)), dicalcium phosphate/starch (for example, 25:75 (w/w)), lactose/mannitol (e.g., 1:1, 1:2, 2:1, 1:3 or 3:1), mannitol/microcrystalline cellulose/aerosol (for example, 70:29:1 or 30:69:1), crospovidone/sodium starch glycolate (for example, 1:1, 1:2, or 1:3), and chitosan/aerosol (for example, 1:1).

In a further preferred embodiment, the co-processed excipient is a mixture of corn starch and pre-gelatinized starch. The corn starch and the pre-gelatinized starch may be mixed in any ratio (based on the total weight of the final mixture). However, preferably the portion of corn starch is more than 50% (w/w), for example 60% (w/w), 70% (w/w), 80% (w/w), 85% (w/w), 90% (w/w) or 95% (w/w). Particularly preferably, the co-processed excipient represents a mixture of 85-95% (w/w) corn starch and 5-15% (w/w) pre-gelatinized starch. For example, such mixture can be prepared by co-spray drying. The latter mixture is commercially available from various suppliers, for example from Colorcon, West Point, Pa., USA marketed under the brand name StarCap 1500. In a specific embodiment, the weight ratio between the crystalline vinorelbine monotartrate and StarCap 1500 is in the range between 1:1 (w/w) and 1:10 (w/w), and preferably between 1:1 (w/w) and 1:5 (w/w).

In a further aspect, the present invention relates to the pharmaceutical composition, and particularly the solid oral dosage form, of the present invention for use in the prevention and/or treatment of cancer. In preferred embodiments, the pharmaceutical composition, and particularly the solid oral dosage form, of the present invention is for use in the prevention and/or treatment of non-small cell lung cancer or breast cancer.

In yet another aspect, the present invention relates to a method for the manufacture of the pharmaceutical composition, and particularly the solid oral dosage form, as defined herein, comprising:

(a) providing crystalline vinorelbine monotartrate; and
(b) formulating the crystalline vinorelbine monotartrate in a solid oral dosage form.

Particularly preferably, the crystalline vinorelbine monotartrate is formulated in pulverized form in a capsule, especially a hard gelatin capsule. Alternatively, the crystalline vinorelbine monotartrate is formulated in a tablet by direct compression or dry compaction. All these techniques are well established in the art.

The invention is further described by the figures and the following examples, which are solely for the purpose of illustrating specific embodiments of this invention, and are not to be construed as limiting the claimed subject matter in any way.

### EXAMPLES

### Materials and Methods

X-ray powder diffraction analysis was performed using a STOE-STADI P transmission diffractometer with the following setup: nCu-Kα₁ radiation (λ=1.54056 Å); U=40 kV; I=35 mA; primary beam monochromator (curved Ge(111)); linear position sensitive detector; slits: 1 mm; d=8 mm; angle region: 2Θ=2 to 38; step width Δ2Θ=0.02°; 25 s/0.2° step.

The powder is originally filled between two Mylar foils and then into the sample holder having a d=8 mm mask.

Thermogravimetric analysis (TGA; for determining the content of residual solvents) was performed by precise sample weighing into alumina crucibles (100 μl, sealed with an alumina lid having a laser drilled 50 μm hole) by using a calibrated ultra-micro balance. Measurement: Mettler TGA/DSC1, large oven; gas control-box (purging gas: N₂, 80 ml/min, mass-flow controlled).

Differential scanning calorimetry (DSC; for determining the melting point) was performed by precise sample weighing into alumina crucibles (70 μl, hermetically closed with an alumina lid) by using a calibrated ultra-micro balance. Measurement: Mettler TC11-TA-Processor with DSC 30 module or Mettler DSC 25 with silver-oven and ceramic sensor crystal and Mettler TC15A-TA-Controller (25° C. to 250° C., 10° C./min). Purging gas: N₂, 80 ml/min, mass-flow controlled. Calibration: Performed directly before the sample measurement with ultrapure Indium (In) as a reference material (temperature scale, heat-flow scale).

HPLC analysis and calculation of the content of impurities in Vinorelbine were performed according to the method described in European Pharmacopoeia 7.0.

### Example 1: Preparation of Crystalline Vinorelbine Monotartrate Acetone Solvate from Vinorelbine Bitartrate

Vinorelbine bitartrate (1000 g) was dissolved in water (10 L) and the pH was adjusted to 6.0 with NaOH. The mixture was treated with CH₂Cl₂ (10 L) and stirring was continued for a further 10 min. The organic phase was separated and treated with water (3 L). Stirring was continued for a further 10 min and the organic phase (8-12 L) was separated. The solvent was evaporated (40° C., 380-400 torr, then down to <25 torr). The residue was dissolved in acetone (7 L). L(+)-tartaric acid in the calculated amount needed for the preparation of vinorelbine monotartrate (according to the titration results) was added. The obtained vinorelbine monotartrate solution was heated to reflux and stirring was continued for about 1 h. The mixture was concentrated in vacuum (70-100 torr; about 1 L of acetone was evaporated). The resulting mixture was filtered and precipitate was washed with acetone (1 L) and dried in vacuum (40-50° C., 25 torr, 2-4 h). Yield—905 g, HPLC purity—99.9%, acetone content—9.5% (GC(gas chromatography)).

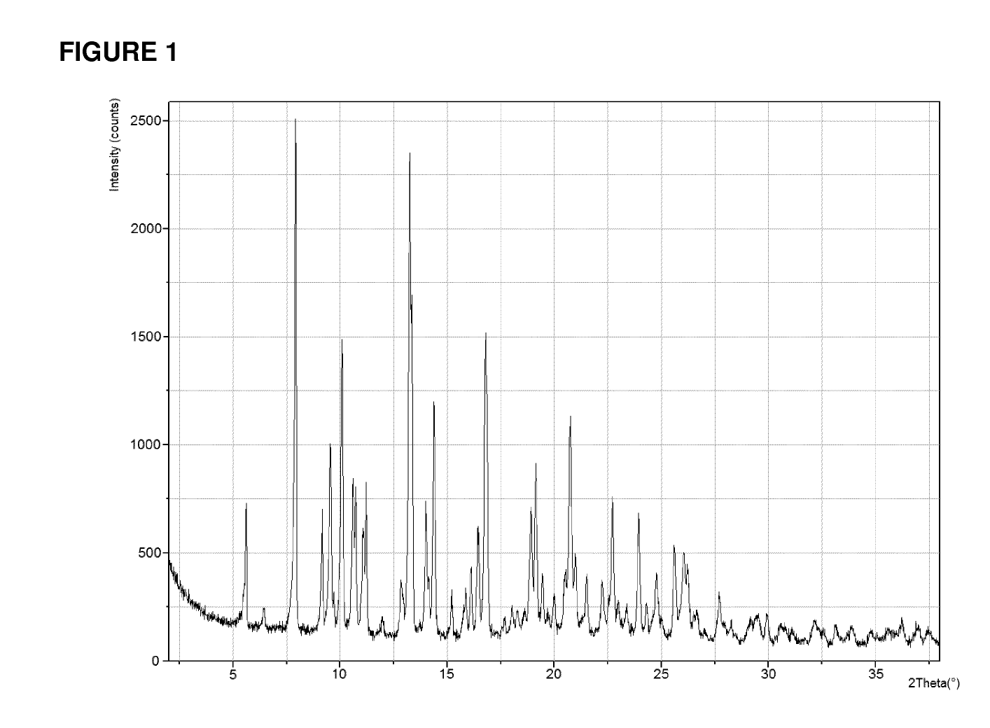

The obtained sample was characterized by powder X-ray diffraction and a PXRD pattern as depicted in FIG. 1 with peaks as listed in Table 3 was obtained.

TABLE 3
PXRD peak table for crystalline vinorelbine
monotartrate acetone solvate

| Pos. | Height | FWHM | d-spacing | Rel. Int. |
|---|---|---|---|---|
| [°2θ] | [cts] | Left [°2θ] | [Å] | [%] |
| 5.6054 | 546.07 | 0.0512 | 15.76664 | 24.18 |
| 6.4609 | 83.64 | 0.0768 | 13.68074 | 3.70 |
| 7.9212 | 2258.58 | 0.0640 | 11.16156 | 100.00 |
| 9.1633 | 517.82 | 0.0512 | 9.65126 | 22.93 |
| 9.5573 | 821.26 | 0.0895 | 9.25423 | 36.36 |
| 9.7065 | 184.16 | 0.0512 | 9.11228 | 8.15 |
| 10.0853 | 1320.51 | 0.0895 | 8.77085 | 58.47 |
| 10.5932 | 685.83 | 0.0768 | 8.35144 | 30.37 |
| 10.7238 | 638.00 | 0.0512 | 8.25002 | 28.25 |
| 11.0545 | 445.22 | 0.0768 | 8.00396 | 19.71 |
| 11.2152 | 693.81 | 0.0640 | 7.88965 | 30.72 |
| 11.9540 | 74.17 | 0.0640 | 7.40367 | 3.28 |
| 12.8359 | 245.98 | 0.0512 | 6.89688 | 10.89 |
| 13.2416 | 2197.05 | 0.0768 | 6.68650 | 97.28 |
| 13.3568 | 1426.23 | 0.0512 | 6.62909 | 63.15 |
| 13.7171 | 54.85 | 0.0768 | 6.45574 | 2.43 |
| 13.9961 | 587.62 | 0.0768 | 6.32768 | 26.02 |
| 14.1349 | 255.75 | 0.0512 | 6.26586 | 11.32 |
| 14.3848 | 1055.76 | 0.0895 | 6.15758 | 46.74 |
| 15.2099 | 196.42 | 0.0768 | 5.82535 | 8.70 |
| 15.8701 | 210.95 | 0.0512 | 5.58447 | 9.34 |
| 16.1017 | 308.06 | 0.0768 | 5.50465 | 13.64 |
| 16.4361 | 504.84 | 0.0895 | 5.39341 | 22.35 |
| 16.7608 | 1278.60 | 0.0895 | 5.28964 | 56.61 |
| 16.8570 | 1051.35 | 0.0512 | 5.25966 | 46.55 |
| 17.6794 | 74.40 | 0.0768 | 5.01683 | 3.29 |
| 18.0195 | 133.67 | 0.0640 | 4.92289 | 5.92 |
| 18.2914 | 116.58 | 0.1023 | 4.85033 | 5.16 |
| 18.6251 | 115.81 | 0.0895 | 4.76417 | 5.13 |
| 18.9071 | 585.25 | 0.0895 | 4.69374 | 25.91 |
| 19.1306 | 798.17 | 0.0895 | 4.63940 | 35.34 |
| 19.4524 | 278.90 | 0.0895 | 4.56336 | 12.35 |
| 19.7038 | 100.61 | 0.0768 | 4.50572 | 4.45 |
| 19.9887 | 181.38 | 0.1151 | 4.44214 | 8.03 |
| 20.5121 | 286.29 | 0.0640 | 4.32996 | 12.68 |
| 20.7307 | 945.30 | 0.1023 | 4.28478 | 41.85 |
| 21.0029 | 341.46 | 0.0895 | 4.22987 | 15.12 |
| 21.5075 | 284.46 | 0.0895 | 4.13176 | 12.59 |
| 22.2002 | 231.05 | 0.0640 | 4.00438 | 10.23 |
| 22.7048 | 629.50 | 0.1023 | 3.91651 | 27.87 |
| 22.9812 | 159.40 | 0.0768 | 3.87003 | 7.06 |
| 23.3779 | 130.97 | 0.1023 | 3.80524 | 5.80 |
| 23.9488 | 523.75 | 0.1151 | 3.71581 | 23.19 |
| 24.2961 | 166.68 | 0.0640 | 3.66347 | 7.38 |
| 24.7640 | 299.93 | 0.0895 | 3.59531 | 13.28 |
| 25.0276 | 89.12 | 0.0768 | 3.55804 | 3.95 |
| 25.5884 | 432.27 | 0.1151 | 3.48132 | 19.14 |
| 26.0488 | 397.87 | 0.1151 | 3.42083 | 17.62 |
| 26.2298 | 313.05 | 0.1023 | 3.39763 | 13.86 |
| 26.6679 | 117.47 | 0.1279 | 3.34280 | 5.20 |
| 27.6944 | 209.73 | 0.1151 | 3.22119 | 9.29 |
| 28.2577 | 83.62 | 0.0768 | 3.15825 | 3.70 |
| 29.1130 | 76.89 | 0.2047 | 3.06737 | 3.40 |
| 29.5362 | 113.22 | 0.1279 | 3.02438 | 5.01 |
| 29.9278 | 117.80 | 0.1279 | 2.98569 | 5.22 |
| 30.4595 | 65.59 | 0.0468 | 2.93234 | 2.90 |
| 30.5471 | 63.69 | 0.1535 | 2.92656 | 2.82 |
| 31.0502 | 32.60 | 0.0768 | 2.88028 | 1.44 |
| 32.1358 | 95.29 | 0.1535 | 2.78542 | 4.22 |
| 33.1254 | 73.27 | 0.1535 | 2.70443 | 3.24 |
| 33.9048 | 68.75 | 0.1535 | 2.64403 | 3.04 |
| 34.7395 | 45.37 | 0.2303 | 2.58238 | 2.01 |
| 35.5866 | 52.92 | 0.2047 | 2.52283 | 2.34 |
| 35.9330 | 37.32 | 0.4093 | 2.49930 | 1.65 |
| 36.2278 | 106.54 | 0.0768 | 2.47964 | 4.72 |
| 36.9643 | 64.14 | 0.2558 | 2.43191 | 2.84 |
| 37.4205 | 50.27 | 0.2558 | 2.40330 | 2.23 |

### Example 2: Preparation of Crystalline Vinorelbine Monotartrate Diethyl Ketone Solvate

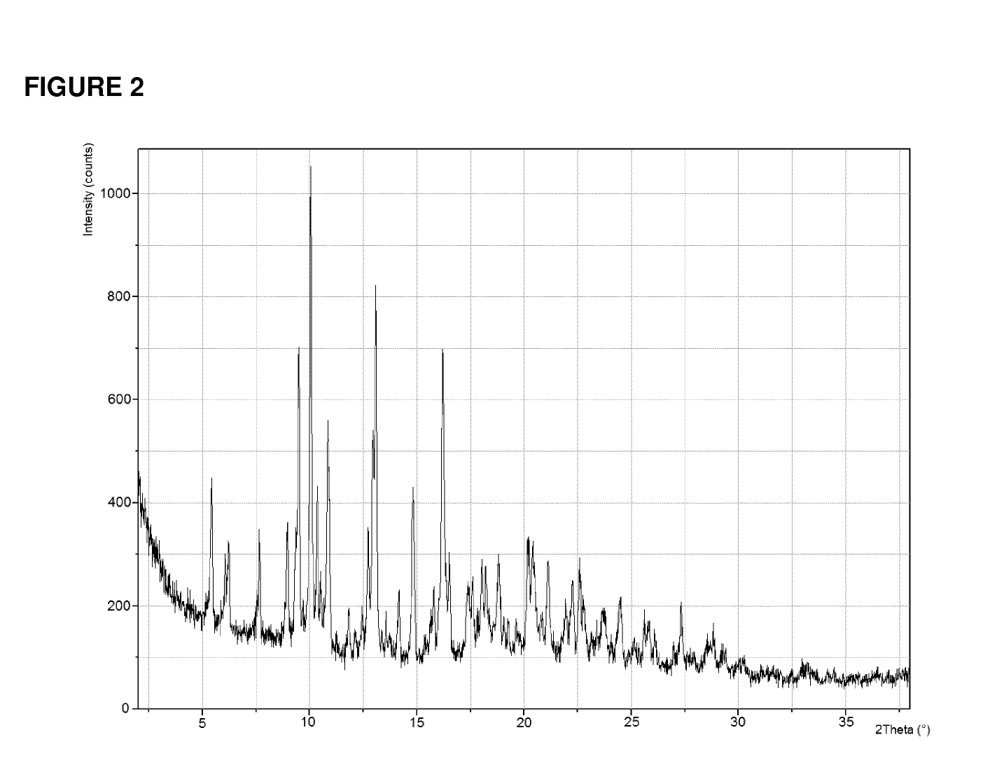

1.5 g of vinorelbine monotartrate acetone solvate as prepared in example 1 was dissolved in 20 mL of dichlomethane. The resulting solution was evaporated to dryness under reduced pressure at 40° C. The residue was dissolved in 17 mL of diethyl ketone under stirring at 40° C. The resulting mixture was stirred for 2 hours at 50-55° C. until the crystallization completed. After cooling to room temperature the crystals were filtered, washed with diethyl ketone and dried under vacuum for 2 hours at about 55° C. 1.4 g of vinorelbine monotartrate diethyl ketone solvate with HPLC purity—99.9% and diethyl ketone content—9.4% (GC) was obtained. The obtained sample was characterized by powder X-ray diffraction and a PXRD pattern as depicted in FIG. 2 with peaks as listed in Table 4 was obtained.

TABLE 4
PXRD peak table for crystalline vinorelbine
monotartrate diethyl ketone solvate

| Pos. | Height | FWHM | d-spacing | Rel. Int. |
|---|---|---|---|---|
| [°2θ] | [cts] | Left [°2θ] | [Å] | [%] |
| 5.4322 | 238.16 | 0.0895 | 16.26880 | 26.12 |
| 6.0603 | 124.60 | 0.0384 | 14.58412 | 13.67 |
| 6.2281 | 160.05 | 0.0768 | 14.19148 | 17.55 |
| 7.6486 | 169.84 | 0.0640 | 11.55881 | 18.63 |
| 8.9803 | 207.68 | 0.0895 | 9.84752 | 22.78 |
| 9.5121 | 481.78 | 0.0895 | 9.29809 | 52.84 |
| 10.0506 | 911.81 | 0.0768 | 8.80112 | 100.00 |
| 10.3530 | 299.51 | 0.0512 | 8.54473 | 32.85 |
| 10.5087 | 114.56 | 0.0640 | 8.41843 | 12.56 |
| 10.8324 | 422.95 | 0.0768 | 8.16755 | 46.39 |
| 11.8135 | 71.28 | 0.0895 | 7.49142 | 7.82 |
| 12.1053 | 22.47 | 0.1023 | 7.31143 | 2.46 |
| 12.4340 | 66.60 | 0.0384 | 7.11892 | 7.30 |
| 12.7216 | 212.76 | 0.0640 | 6.95863 | 23.33 |
| 12.9317 | 391.81 | 0.0512 | 6.84604 | 42.97 |
| 13.0902 | 678.07 | 0.0768 | 6.76350 | 74.36 |
| 13.5542 | 49.81 | 0.0512 | 6.53296 | 5.46 |
| 14.1548 | 112.50 | 0.0768 | 6.25710 | 12.34 |
| 14.8028 | 288.34 | 0.0895 | 5.98463 | 31.62 |
| 15.6304 | 46.90 | 0.2047 | 5.66957 | 5.14 |
| 15.7775 | 112.78 | 0.0768 | 5.61701 | 12.37 |
| 16.2191 | 568.21 | 0.0895 | 5.46508 | 62.32 |
| 16.5148 | 195.24 | 0.0768 | 5.36787 | 21.41 |
| 17.3592 | 121.62 | 0.0895 | 5.10863 | 13.34 |
| 17.5835 | 140.36 | 0.0640 | 5.04396 | 15.39 |
| 18.0223 | 178.60 | 0.0895 | 4.92214 | 19.59 |
| 18.2024 | 175.47 | 0.0512 | 4.87384 | 19.24 |
| 18.8281 | 171.61 | 0.1279 | 4.71326 | 18.82 |
| 19.2623 | 72.49 | 0.0768 | 4.60797 | 7.95 |
| 19.6168 | 70.39 | 0.0768 | 4.52551 | 7.72 |
| 20.1526 | 226.77 | 0.0512 | 4.40638 | 24.87 |
| 20.3930 | 216.82 | 0.0768 | 4.35497 | 23.78 |
| 20.8221 | 76.12 | 0.1023 | 4.26618 | 8.35 |
| 21.1076 | 193.31 | 0.0895 | 4.20913 | 21.20 |
| 21.9443 | 110.20 | 0.0768 | 4.05049 | 12.09 |
| 22.2489 | 155.79 | 0.0768 | 3.99572 | 17.09 |
| 22.5867 | 178.45 | 0.1023 | 3.93672 | 19.57 |
| 22.7677 | 116.05 | 0.0768 | 3.90584 | 12.73 |
| 23.1250 | 54.75 | 0.0768 | 3.84629 | 6.00 |
| 23.6966 | 96.70 | 0.2047 | 3.75478 | 10.60 |
| 24.4980 | 119.33 | 0.1279 | 3.63374 | 13.09 |
| 25.1266 | 53.78 | 0.1535 | 3.54424 | 5.90 |
| 25.3505 | 41.65 | 0.0640 | 3.51344 | 4.57 |
| 25.5926 | 87.65 | 0.0768 | 3.48075 | 9.61 |
| 25.8438 | 83.76 | 0.1279 | 3.44750 | 9.19 |
| 26.0984 | 60.49 | 0.0768 | 3.41443 | 6.63 |
| 26.9579 | 53.91 | 0.0384 | 3.30749 | 5.91 |
| 27.3227 | 129.77 | 0.0384 | 3.26416 | 14.23 |
| 28.8302 | 68.84 | 0.0768 | 3.09682 | 7.55 |
| 29.3425 | 37.47 | 0.3070 | 3.04390 | 4.11 |
| 30.1656 | 22.61 | 0.3070 | 2.96269 | 2.48 |
| 31.4341 | 5.83 | 0.6140 | 2.84597 | 0.64 |
| 33.0439 | 16.97 | 0.3070 | 2.71091 | 1.86 |
| 34.2809 | 4.58 | 0.4093 | 2.61587 | 0.50 |

### Example 3: Preparation of Crystalline Vinorelbine Monotartrate Ethyl Acetate Solvate

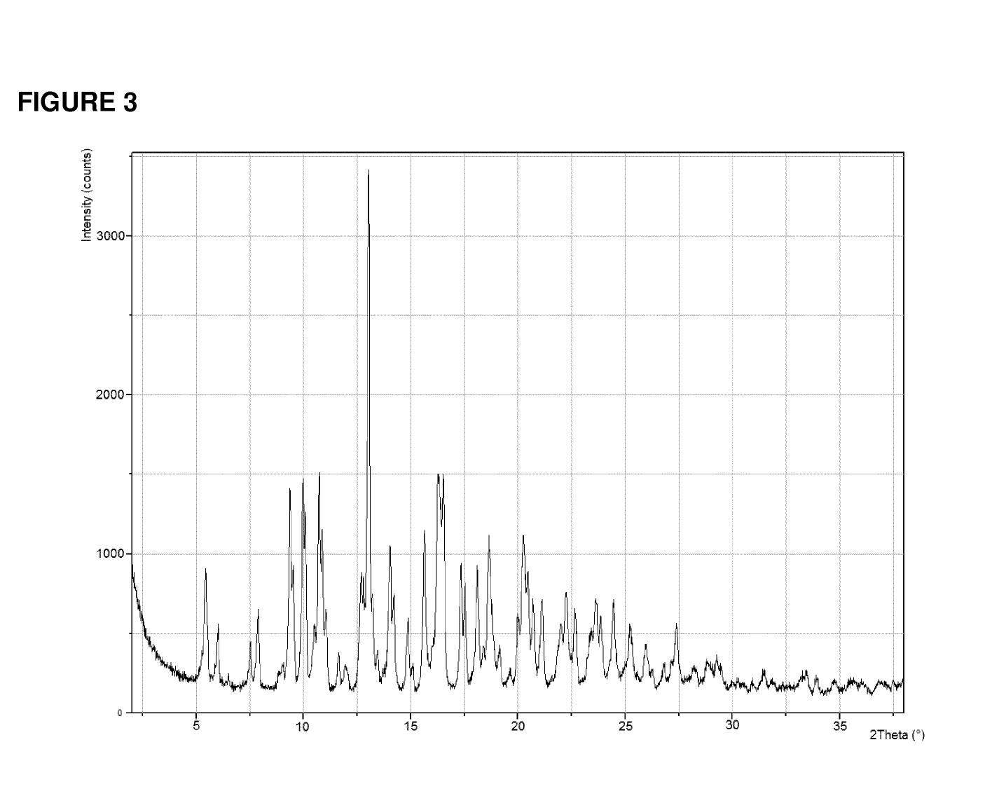

1.5 g of vinorelbine monotartrate acetone solvate as prepared in example 1 was dissolved in 20 mL of dichlomethane. The resulting solution was evaporated to dryness under reduced pressure at 40° C. The residue was dissolved in 40 mL of ethyl acetate under stirring at 40° C. The resulting mixture was stirred for 2 hours at 50-55° C. until the crystallization completed. After cooling to room temperature the crystals were filtered, washed with ethyl acetate and dried under vacuum for 2 hours at about 55° C. 1.2 g of vinorelbine monotartrate ethyl acetate solvate with HPLC purity—99.9% and ethyl acetate content—14.2% (GC) was obtained. The obtained sample was characterized by powder X-ray diffraction and a PXRD pattern as depicted in FIG. 3 with peaks as listed in Table 5 was obtained.

TABLE 5
PXRD peak table for crystalline vinorelbine monotartrate
ethyl acetate solvate

| | Height | FWHM | d-spacing | Rel. |
|---|---|---|---|---|
| Pos. [°2θ] | [cts] | Left [°2θ] | [Å] | Int. [%] |
| 5.4294 | 689.76 | 0.1151 | 16.27716 | 21.31 |
| 6.0307 | 352.73 | 0.0895 | 14.65556 | 10.90 |
| 6.5111 | 34.38 | 0.1023 | 13.57527 | 1.06 |
| 7.5178 | 258.05 | 0.0768 | 11.75957 | 7.97 |
| 7.8826 | 469.71 | 0.0768 | 11.21613 | 14.51 |
| 8.8474 | 84.17 | 0.0768 | 9.99514 | 2.60 |
| 9.0307 | 130.74 | 0.0512 | 9.79260 | 4.04 |
| 9.3732 | 1217.63 | 0.0768 | 9.43553 | 37.61 |
| 9.5284 | 669.55 | 0.0640 | 9.28218 | 20.68 |
| 9.9644 | 1249.54 | 0.0768 | 8.87703 | 38.60 |
| 10.0981 | 1047.17 | 0.0640 | 8.75976 | 32.35 |
| 10.5044 | 380.12 | 0.0640 | 8.42191 | 11.74 |
| 10.7257 | 1308.07 | 0.0768 | 8.24857 | 40.41 |
| 10.8696 | 975.63 | 0.0512 | 8.13975 | 30.14 |
| 11.0511 | 452.46 | 0.1023 | 8.00643 | 13.98 |
| 11.6551 | 186.10 | 0.0895 | 7.59286 | 5.75 |
| 11.9357 | 129.31 | 0.1023 | 7.41496 | 3.99 |
| 12.0576 | 91.63 | 0.0768 | 7.34025 | 2.83 |
| 12.6717 | 657.45 | 0.0640 | 6.98590 | 20.31 |
| 12.7846 | 686.64 | 0.0256 | 6.92444 | 21.21 |
| 13.0371 | 3237.16 | 0.0895 | 6.79092 | 100.00 |
| 13.2157 | 556.86 | 0.0768 | 6.69953 | 17.20 |
| 13.4745 | 213.78 | 0.1023 | 6.57144 | 6.60 |
| 14.0552 | 808.92 | 0.1023 | 6.30122 | 24.99 |
| 14.2248 | 577.55 | 0.0895 | 6.22648 | 17.84 |
| 14.8772 | 420.59 | 0.0768 | 5.95487 | 12.99 |
| 15.0941 | 146.26 | 0.0768 | 5.86977 | 4.52 |
| 15.6461 | 972.99 | 0.1279 | 5.66391 | 30.06 |
| 16.2364 | 1189.45 | 0.0895 | 5.45928 | 36.74 |
| 16.5391 | 1208.19 | 0.0895 | 5.36004 | 37.32 |
| 17.3417 | 767.49 | 0.1023 | 5.11373 | 23.71 |
| 17.5265 | 648.27 | 0.0768 | 5.06025 | 20.03 |
| 18.1034 | 756.87 | 0.1151 | 4.90027 | 23.38 |
| 18.3968 | 254.40 | 0.0640 | 4.82276 | 7.86 |
| 18.6356 | 922.41 | 0.1023 | 4.76149 | 28.49 |
| 19.1410 | 257.45 | 0.0640 | 4.63691 | 7.95 |
| 19.6147 | 98.29 | 0.1279 | 4.52599 | 3.04 |
| 19.9770 | 444.91 | 0.0640 | 4.44471 | 13.74 |
| 20.2478 | 957.12 | 0.0640 | 4.38587 | 29.57 |
| 20.4682 | 725.32 | 0.0640 | 4.33915 | 22.41 |
| 20.7102 | 517.28 | 0.1023 | 4.28898 | 15.98 |
| 21.1079 | 543.29 | 0.1407 | 4.20906 | 16.78 |
| 21.9959 | 399.90 | 0.0768 | 4.04111 | 12.35 |
| 22.2168 | 581.76 | 0.1279 | 4.00142 | 17.97 |
| 22.6634 | 484.58 | 0.1151 | 3.92357 | 14.97 |
| 23.3790 | 324.86 | 0.1279 | 3.80507 | 10.04 |
| 23.6560 | 528.46 | 0.1279 | 3.76114 | 16.32 |
| 23.8446 | 452.32 | 0.0768 | 3.73181 | 13.97 |
| 24.4786 | 527.95 | 0.0768 | 3.63658 | 16.31 |
| 25.2045 | 410.29 | 0.0640 | 3.53346 | 12.67 |
| 25.9496 | 271.22 | 0.1791 | 3.43368 | 8.38 |
| 26.2679 | 109.74 | 0.1023 | 3.39278 | 3.39 |
| 26.8010 | 132.74 | 0.0768 | 3.32650 | 4.10 |
| 27.1254 | 159.67 | 0.0768 | 3.28745 | 4.93 |
| 27.4072 | 387.05 | 0.1151 | 3.25429 | 11.96 |
| 28.3327 | 116.86 | 0.0768 | 3.15005 | 3.61 |
| 28.7856 | 170.72 | 0.1279 | 3.10151 | 5.27 |
| 29.2461 | 193.32 | 0.1023 | 3.05372 | 5.97 |
| 29.4963 | 136.38 | 0.1023 | 3.02838 | 4.21 |
| 29.9778 | 45.20 | 0.1279 | 2.98082 | 1.40 |
| 30.6234 | 17.92 | 0.1791 | 2.91944 | 0.55 |
| 30.8915 | 42.39 | 0.1279 | 2.89472 | 1.31 |
| 31.4969 | 111.69 | 0.1791 | 2.84044 | 3.45 |
| 31.8710 | 42.71 | 0.1535 | 2.80795 | 1.32 |
| 33.4623 | 105.00 | 0.1023 | 2.67797 | 3.24 |
| 33.9398 | 65.06 | 0.1791 | 2.64138 | 2.01 |
| 34.6635 | 42.63 | 0.1279 | 2.58787 | 1.32 |
| 35.5644 | 50.66 | 0.3070 | 2.52436 | 1.56 |
| 36.0214 | 49.30 | 0.2047 | 2.49337 | 1.52 |
| 36.7783 | 46.37 | 0.2047 | 2.44378 | 1.43 |
| 37.4700 | 49.54 | 0.1023 | 2.40024 | 1.53 |

### Example 4: Preparation of Crystalline Vinorelbine Monotartrate Isopropanol Solvate

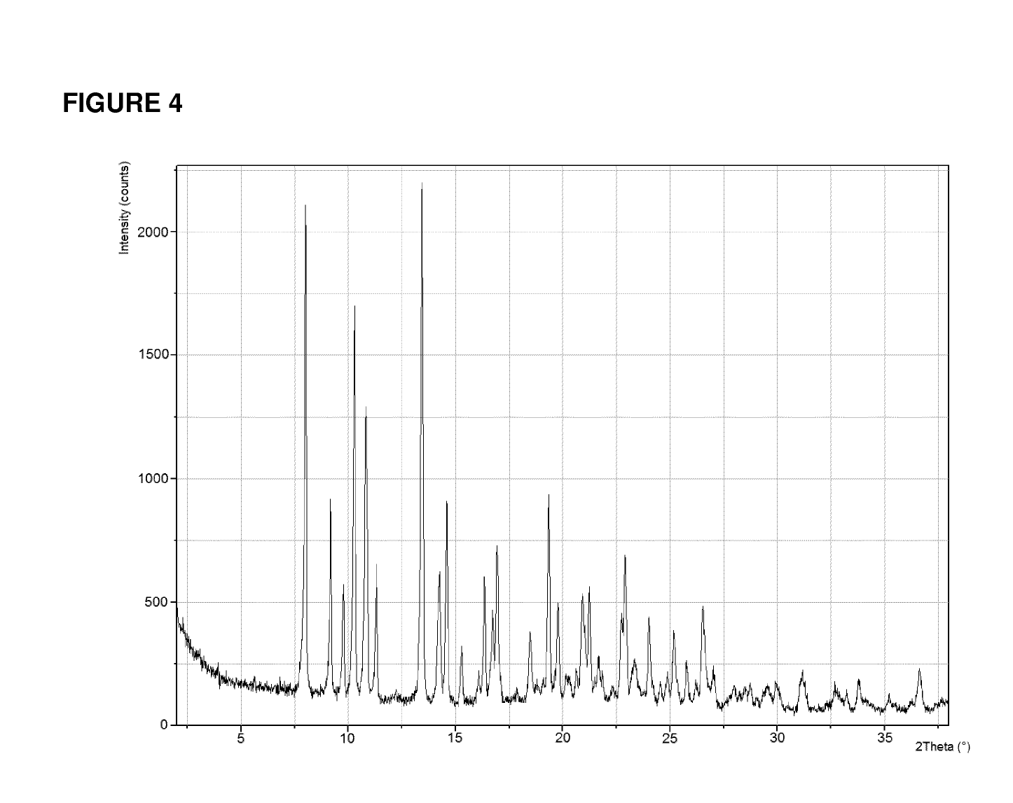

1.5 g of vinorelbine monotartrate acetone solvate as prepared in example 1 was dissolved in 20 mL of dichlomethane. The resulting solution was evaporated to dryness under reduced pressure at 40° C. The residue was dissolved in 40 mL of isopropanol under stirring at 45-50° C. The mixture was slowly evaporated under reduced pressure at 45-50° C. to 50% of the initial volume. The resulting mixture was stirred for 2 hours at 50-55° C. until the crystallization completed. After cooling to room temperature the crystals were filtered, washed with isopropanol and dried under vacuum for 2 hours at about 55° C. 1.3 g of vinorelbine monotartrate isopropanol solvate with HPLC purity—99.9% and isopropanol content—8.4% (GC) was obtained. The obtained sample was characterized by powder X-ray diffraction and a PXRD pattern as depicted in FIG. 4 with peaks as listed in Table 6 was obtained.

TABLE 6
PXRD peak table for crystalline vinorelbine
monotartrate isopropanol solvate

| Pos. | Height | FWHM | d-spacing | Rel. |
|---|---|---|---|---|
| [°2θ] | [cts] | Left [°2θ] | [Å] | Int. [%] |
| 8.0129 | 1903.57 | 0.0640 | 11.03407 | 91.93 |
| 9.1809 | 749.59 | 0.0640 | 9.63282 | 36.20 |
| 9.7761 | 424.06 | 0.0768 | 9.04761 | 20.48 |
| 10.2899 | 1571.44 | 0.0768 | 8.59692 | 75.89 |
| 10.8003 | 1145.83 | 0.0768 | 8.19182 | 55.34 |
| 10.8819 | 791.14 | 0.0384 | 8.13053 | 38.21 |
| 11.3103 | 521.37 | 0.0640 | 7.82351 | 25.18 |
| 12.2000 | 18.07 | 0.3070 | 7.25490 | 0.87 |
| 13.4317 | 2070.70 | 0.1151 | 6.59229 | 100.00 |
| 14.2650 | 512.67 | 0.0768 | 6.20902 | 24.76 |
| 14.5880 | 804.73 | 0.0895 | 6.07225 | 38.86 |
| 15.2858 | 210.61 | 0.0768 | 5.79660 | 10.17 |
| 16.0847 | 117.63 | 0.0512 | 5.51042 | 5.68 |
| 16.3481 | 501.44 | 0.0768 | 5.42225 | 24.22 |
| 16.7267 | 360.77 | 0.0640 | 5.30034 | 17.42 |
| 16.9188 | 615.54 | 0.0895 | 5.24060 | 29.73 |
| 17.8557 | 46.11 | 0.1535 | 4.96768 | 2.23 |
| 18.4670 | 281.01 | 0.1279 | 4.80460 | 13.57 |
| 19.3367 | 821.47 | 0.1151 | 4.59041 | 39.67 |
| 19.7750 | 407.57 | 0.0895 | 4.48965 | 19.68 |
| 20.1040 | 86.52 | 0.0895 | 4.41691 | 4.18 |
| 20.6233 | 138.24 | 0.1023 | 4.30686 | 6.68 |
| 20.9202 | 438.44 | 0.1279 | 4.24640 | 21.17 |
| 21.2237 | 439.89 | 0.0895 | 4.18636 | 21.24 |
| 21.6569 | 184.35 | 0.1023 | 4.10358 | 8.90 |
| 21.8446 | 116.57 | 0.0512 | 4.06875 | 5.63 |
| 22.3243 | 68.95 | 0.1535 | 3.98239 | 3.33 |
| 22.7100 | 324.34 | 0.0768 | 3.91562 | 15.66 |
| 22.8892 | 559.79 | 0.1279 | 3.88537 | 27.03 |
| 23.3789 | 163.57 | 0.1279 | 3.80508 | 7.90 |
| 23.9938 | 338.01 | 0.0895 | 3.70895 | 16.32 |
| 24.5439 | 89.84 | 0.1279 | 3.62705 | 4.34 |
| 24.8749 | 130.45 | 0.0640 | 3.57954 | 6.30 |
| 25.1658 | 301.50 | 0.1151 | 3.53881 | 14.56 |
| 25.7526 | 183.09 | 0.0512 | 3.45949 | 8.84 |
| 26.1873 | 102.54 | 0.1023 | 3.40305 | 4.95 |
| 26.5238 | 406.86 | 0.1023 | 3.36064 | 19.65 |
| 27.0243 | 144.78 | 0.1279 | 3.29952 | 6.99 |
| 27.9588 | 74.02 | 0.1279 | 3.19132 | 3.57 |
| 28.4395 | 77.97 | 0.1279 | 3.13847 | 3.77 |
| 28.7491 | 84.79 | 0.1279 | 3.10537 | 4.09 |
| 29.5508 | 87.79 | 0.3070 | 3.02292 | 4.24 |
| 29.9281 | 103.23 | 0.1023 | 2.98566 | 4.99 |
| 31.1594 | 137.20 | 0.1023 | 2.87044 | 6.63 |
| 32.6595 | 76.23 | 0.1535 | 2.74194 | 3.68 |
| 33.2338 | 62.45 | 0.1023 | 2.69586 | 3.02 |
| 33.7939 | 106.13 | 0.1279 | 2.65245 | 5.13 |
| 35.2115 | 58.01 | 0.1023 | 2.54884 | 2.80 |
| 36.2464 | 20.06 | 0.2047 | 2.47841 | 0.97 |
| 36.6244 | 156.76 | 0.1023 | 2.45369 | 7.57 |

### Example 5: Preparation of Crystalline Vinorelbine Monotartrate Hydrate

150 g of vinorelbine monotartrate acetone solvate obtained as described in example 1 was incubated at 60° C. and a relative humidity of about 40% for 16 hours. Finally, 140 g of crystalline vinorelbine monotartrate hydrate with HPLC purity—99.9% and residual acetone content—0.16% (GC) was obtained. The obtained sample was characterized by powder X-ray diffraction and a PXRD pattern as depicted in FIG. 5 with peaks as listed in Table 7 was obtained.

TABLE 7
PXRD peak table for crystalline vinorelbine
monotartrate hydrate

| Pos. | Height | FWHM | d-spacing | Rel. |
|---|---|---|---|---|
| [°2θ] | [cts] | Left [°2θ] | [Å] | Int. [%] |
| 5.6603 | 155.39 | 0.0640 | 15.61370 | 9.70 |
| 6.3047 | 64.91 | 0.0640 | 14.01933 | 4.05 |
| 7.8920 | 1601.52 | 0.0640 | 11.20280 | 100.00 |
| 9.5022 | 619.80 | 0.0768 | 9.30778 | 38.70 |
| 9.6400 | 526.70 | 0.0512 | 9.17505 | 32.89 |
| 10.3129 | 1349.49 | 0.1151 | 8.57780 | 84.26 |
| 10.7438 | 1179.93 | 0.0895 | 8.23476 | 73.68 |
| 10.8241 | 803.11 | 0.0640 | 8.17386 | 50.15 |
| 11.0432 | 149.93 | 0.0640 | 8.01218 | 9.36 |
| 11.4028 | 398.03 | 0.0895 | 7.76029 | 24.85 |
| 12.0440 | 46.35 | 0.0768 | 7.34854 | 2.89 |
| 13.4144 | 1450.23 | 0.0640 | 6.60074 | 90.55 |
| 13.5607 | 1344.16 | 0.0895 | 6.52987 | 83.93 |
| 13.9826 | 253.57 | 0.1279 | 6.33378 | 15.83 |
| 14.1259 | 211.20 | 0.0512 | 6.26981 | 13.19 |
| 14.5737 | 764.07 | 0.1151 | 6.07818 | 47.71 |
| 15.4909 | 82.37 | 0.0640 | 5.72031 | 5.14 |
| 15.8024 | 114.03 | 0.1023 | 5.60824 | 7.12 |
| 16.2052 | 242.01 | 0.0895 | 5.46972 | 15.11 |
| 16.5319 | 99.57 | 0.0512 | 5.36236 | 6.22 |
| 17.1502 | 389.84 | 0.0640 | 5.17042 | 24.34 |
| 17.2971 | 364.86 | 0.0640 | 5.12684 | 22.78 |
| 18.3869 | 191.25 | 0.1535 | 4.82534 | 11.94 |
| 18.9855 | 427.01 | 0.0768 | 4.67454 | 26.66 |
| 19.4119 | 655.89 | 0.0640 | 4.57280 | 40.95 |
| 19.5577 | 490.86 | 0.0512 | 4.53904 | 30.65 |
| 20.0486 | 75.58 | 0.2558 | 4.42900 | 4.72 |
| 20.7169 | 244.33 | 0.0640 | 4.28761 | 15.26 |
| 21.1602 | 278.41 | 0.1023 | 4.19877 | 17.38 |
| 21.4709 | 224.42 | 0.0768 | 4.13871 | 14.01 |
| 22.2069 | 220.45 | 0.0640 | 4.00318 | 13.76 |
| 22.7214 | 119.84 | 0.1023 | 3.91369 | 7.48 |
| 23.1938 | 350.58 | 0.0512 | 3.83504 | 21.89 |
| 23.5706 | 93.54 | 0.1279 | 3.77457 | 5.84 |
| 24.1472 | 87.34 | 0.0768 | 3.68573 | 5.45 |
| 24.7625 | 234.71 | 0.1535 | 3.59552 | 14.66 |
| 25.1137 | 105.37 | 0.1535 | 3.54604 | 6.58 |
| 25.7867 | 191.69 | 0.0895 | 3.45499 | 11.97 |
| 26.4846 | 207.94 | 0.1535 | 3.36551 | 12.98 |
| 26.8187 | 116.81 | 0.1279 | 3.32434 | 7.29 |
| 27.4412 | 100.78 | 0.3582 | 3.25033 | 6.29 |
| 28.2760 | 47.50 | 0.3070 | 3.15624 | 2.97 |
| 28.7915 | 81.31 | 0.2558 | 3.10089 | 5.08 |
| 29.4985 | 48.48 | 0.1535 | 3.02816 | 3.03 |
| 30.3843 | 100.92 | 0.0768 | 2.94186 | 6.30 |
| 30.8031 | 46.71 | 0.1535 | 2.90282 | 2.92 |
| 32.2225 | 32.43 | 0.1535 | 2.77812 | 2.03 |
| 32.8210 | 16.73 | 0.2047 | 2.72881 | 1.04 |
| 33.3521 | 10.14 | 0.1535 | 2.68656 | 0.63 |
| 34.3526 | 60.81 | 0.1791 | 2.61058 | 3.80 |
| 35.1208 | 29.33 | 0.2558 | 2.55522 | 1.83 |
| 36.1536 | 42.01 | 0.1535 | 2.48456 | 2.62 |

### Example 6: Preparation of Crystalline Vinorelbine Monotartrate Hydrate from Ethyl Acetate Solvate

1.0 g of vinorelbine monotartrate ethyl acetate solvate obtained as described in example 3 was incubated at 30° C. and a relative humidity of about 60% for 120 hours. Finally, 0.9 g of crystalline vinorelbine monotartrate hydrate with HPLC purity—99.9% and residual ethyl acetate content—0.05% (GC) was obtained. The obtained sample was characterized by powder X-ray diffraction and a PXRD pattern is the same as for vinorelbine monotartrate hydrate obtained from acetone solvate depicted in FIG. 5.

### Example 7: Preparation of Crystalline Vinorelbine Monotartrate Hydrate by Crystallization from Wet Ethanol

1.5 g of vinorelbine monotartrate acetone solvate as prepared in example 1 was dissolved in 20 mL of dichlomethane. The resulting solution was evaporated to dryness under reduced pressure at 40° C. The residue was dissolved in 30 mL of absolute ethanol under stirring at 40° C. The obtained solution was vacuum evaporated to 10 mL and 0.2 mL of water was added. Afterwards 20 mg of seeds of crystalline vinorelbine monotartrate hydrate were added and the resulting mixture was stirred for 2 hours at room temperature. The precipitate formed was filtered, washed with absolute ethanol and dried under vacuum for 1 hour at about 55° C. Finally, 0.8 g of crystalline vinorelbine monotartrate hydrate with HPLC purity—99.9% was obtained. The obtained sample was characterized by powder X-ray diffraction and a PXRD pattern is the same as for vinorelbine monotartrate hydrate obtained according to example 5 depicted in FIG. 5.

### Example 8: Stability of the Crystalline Vinorelbine Monotartrate Organic Solvates

Three further representative batches of crystalline vinorelbine monotartrate acetone solvate being produced according to Example 1 were analyzed for stability (i.e. batches no. 011213, no. 021213, and no. 010414). The samples were exposed to temperatures of 5° C. and 25° C. for three and six months, respectively. Batch no. 010414 was also exposed to a temperature of 40° C. (at 60%±2% relative humidity) for 15 days, 1 month, and two months, respectively.

Exemplary results are summarized in the following Table 8.

TABLE 8
Stability data of crystalline vinorelbine monotartrate acetone
solvate at 5° C., 25° C., and 40° C.

| | Degradation products (%) | | |
|---|---|---|---|
| | Photodegradation | Thermodegradation | |
| | (3.6-epoxy | (4-deacetyl | |
| | vinorelbine) | vinorelbine) | Total |
| Specification limits | NMT 0.15 | NMT 0.15 | NMT 0.70 |
| batch no. 011213 | | storage conditions 5° C. | |
| initial | LT 0.05 | 0.06 | 0.14 |
| 3 months | 0.07 | 0.05 | 0.15 |
| 6 months | LT 0.05 | 0.06 | 0.15 |
| batch no. 021213 | | storage conditions 5° C. | |
| initial | LT 0.05 | 0.06 | 0.06 |
| 3 months | LT 0.05 | 0.08 | 0.08 |
| 6 months | LT 0.05 | 0.09 | 0.09 |
| batch no. 010414 | | storage conditions 5° C. | |
| initial | LT 0.05 | LT 0.05 | 0.07 |
| 3 months | 0.07 | LT 0.05 | 0.07 |
| 6 months | LT 0.05 | LT 0.05 | 0.07 |
| batch no. 011213 | | storage conditions 25° C. | |
| initial | LT 0.05 | 0.06 | 0.14 |
| 3 months | LT 0.05 | 0.06 | 0.16 |
| 6 months | LT 0.05 | 0.06 | 0.16 |
| batch no. 021213 | | storage conditions 25° C. | |
| initial | LT 0.05 | 0.06 | 0.06 |
| 3 months | 0.06 | 0.07 | 0.07 |
| 6 months | LT 0.05 | 0.07 | 0.07 |
| batch no. 010414 | | storage conditions 25° C. | |
| initial | LT 0.05 | LT 0.05 | 0.07 |
| 3 months | LT 0.05 | LT 0.05 | 0.06 |
| 6 months | LT 0.05 | LT 0.05 | 0.08 |
| batch no. 010414 | | storage conditions 40° C. | |
| initial | LT 0.05 | LT 0.05 | 0.07 |
| 1 month | LT 0.05 | LT 0.05 | 0.08 |
| 2 months | 0.05 | LT 0.05 | 0.13 |

All three batches of crystalline vinorelbine monotartrate acetone solvate tested exhibited almost no degradation after six months storage both at 5° C.+3° C. and 25° C.±2° C., and only minimal degradation after two months storage at 40° C.

Hence, the stability of crystalline vinorelbine monotartrate solvates is significantly improved as compared to vinorelbine bitartrate as shown in Table 9.

TABLE 9
Comparable stressing stability study of Vinorelbine Monotartrate
organic solvates and Vinorelbine Bitartrate at 60° C.

| | Total degradation impurities, % | | |
|---|---|---|---|
| | Vinorelbine | Vinorelbine | |
| Time | Monotartrate | Monotartrate | Vinorelbine |
| (weeks) | acetone solvate | isopropanol solvate | Bitartrate |
| 1 | LT 0.05 | LT 0.05 | 9.8 |
| 3 | 0.08 | 0.05 | — |
| 8 | 0.24 | 0.15 | — |

### Example 9: Stability of Crystalline Vinorelbine Monotartrate Hydrate

Crystalline vinorelbine monotartrate hydrate being produced according to Example 5 were analyzed for stability. The samples were exposed to temperatures of 25° C. and 40° C. for three and six months, respectively.

The overall accumulation of degradation impurities for crystalline vinorelbine monotartrate hydrate does not exceed 0.15% after 6 months at 40° C. and 0.02% at 25° C. (Table 10).

TABLE 10
Stability data of crystalline vinorelbine monotartrate hydrate at
25° C., and 40° C.

| | Degradation products (%) | | |
|---|---|---|---|
| | Photodegradation | Thermodegradation | |
| | (3,6-epoxy | (4-deacetyl | |
| | vinorelbine) | vinorelbine) | Total |
| Specification limits | NMT 0.15 | NMT 0.15 | NMT 0.70 |
| batch no. 010615 | | storage conditions 25° C. | |
| initial | 0.05 | LT 0.05 | 0.05 |
| 3 months | 0.05 | LT0.05 | 0.05 |
| 6 months | 0.05 | LT0.05 | 0.06 |
| batch no. 010615 | | storage conditions 40° C. | |
| initial | 0.05 | LT 0.05 | 0.05 |
| 3 months | 0.05 | 0.06 | 0.13 |
| 6 months | 0.07 | 0.07 | 0.16 |

Furthermore, a comparative photo-degradation analysis of crystalline vinorelbine monotartrate hydrate, according to the present invention and vinorelbine bitartrate was performed.

The samples (about 14 mg each) were placed in 10 ml light glass volumetric flasks and exposed in a photo chamber to a xenon lamp (wave length 300-800 nm; fluence rate 250-765 W/m²). The amount of the known photo-degradation product 3,6-epoxy vinorelbine was determined at various time points by means of HPLC.

The obtained results are shown in Table 11. The peak due to the known photo degradation product (3,6-Epoxy vinorelbine) was detected in the chromatograms obtained with all the solutions of the illuminated samples. The observed accumulation of 3,6-epoxy vinorelbine was significantly more intense in vinorelbine bitartrate.

TABLE 11
Photo degradation products accumulation during illumination of samples of
vinorelbine bitartrate and crystalline vinorelbine monotartrate hydrate

| | Photodegradation product | | Total impurities (excluding | |
|---|---|---|---|---|
| | (3,6-epoxy vinorelbine), % | | photodegradation product), % | |
| | | Vinorelbine | | Vinorelbine |
| Exposure. | Vinorelbine | Monotartrate | Vinorelbine | Monotartrate |
| min | Bitartrate | Hydrate | Bitartrate | Hydrate |
| 0 | 0.05 | 0.03 | 0.10 | 0.10 |
| 15 | 0.16 | 0.08 | 0.10 | 0.10 |
| 30 | 0.24 | 0.14 | 0.16 | 0.10 |
| 60 | 0.35 | 0.28 | 0.20 | 0.10 |
| 120 | 0.72 | 0.44 | 0.27 | 0.17 |

### Example 10: Preparation of Amorphous Vinorelbine Monotartrate

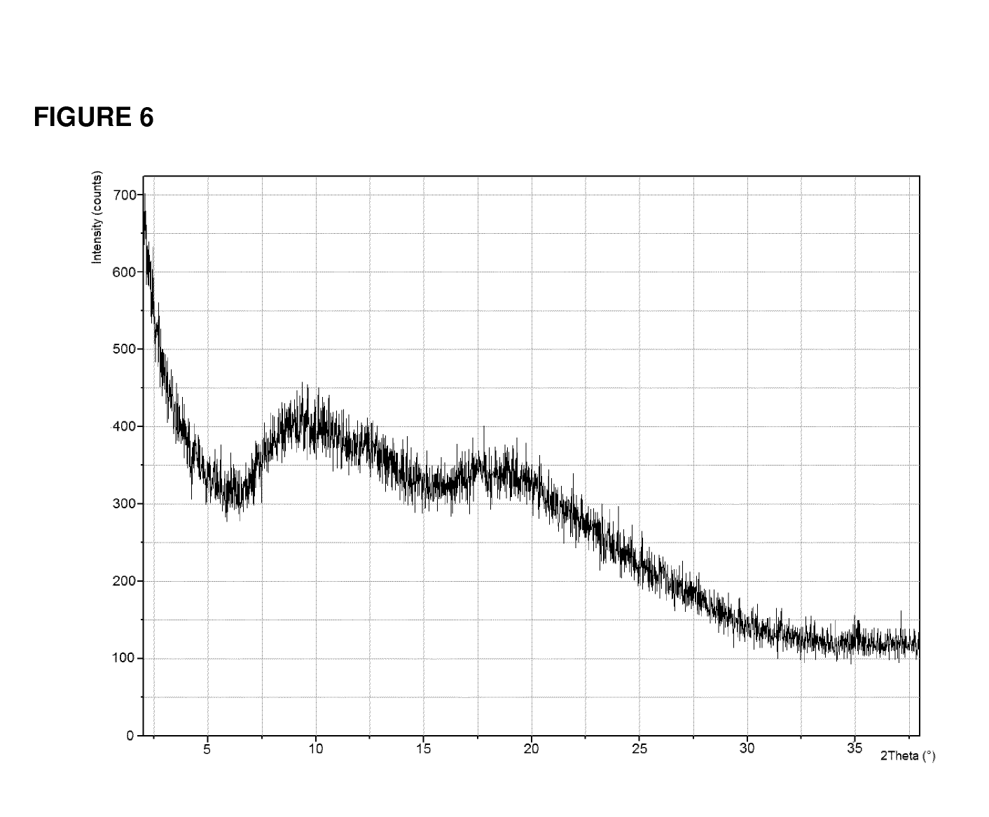

As a comparative example, amorphous vinorelbine monotartrate was prepared according to the following procedure: 2.0 g of vinorelbine monotartrate was dissolved in 5 ml of dichloromethane (DCM) and evaporated to dryness in vacuum at 40° C. for 30 min. Then, the residue was dissolved in 5 ml of DCM. The solution was added to 50 ml of heptane and stirred for about 5 min. The precipitate was filtered, washed with heptane, and dried at 40° C. in vacuum for 20 min. Finally, the sample was analyzed by HPLC. FIG. 6 depicts the results of an exemplary X-ray powder diffraction analysis for representative batch of amorphous vinorelbine monotartrate. No peaks are detectable.

In order to evaluate the thermostability and photostability of the compound the sample was exposed to a temperature of 40° C. for 2 weeks analyzed by HPLC. The results are summarized in Table 12.

TABLE 12
Stability data of amorphous vinorelbine monotartrate at 40° C.

| | Degradation products (%) | | |
|---|---|---|---|
| | 3,6-epoxy | vinorelbine | |
| | vinorelbine | N-oxide | Total |
| Specification limits | NMT 0.15 | NMT 0.15 | NMT 0.70 |
| initial | LT 0.05 | LT 0.05 | 0.13 |
| 15 days | 0.13 | 0.33 | 1.08 |

From the data it is apparent that the amorphous vinorelbine monotartrate (in contrast to the crystalline form of the present invention) exhibits significant degradation already after incubation for two weeks at 40° C. Accordingly, the improved thermo- and photostability data shown above can be specifically assigned to the crystalline form of vinorelbine monotartrate according to the present invention.

### Example 11. Preparation of Vinorelbine Monotartrate Acetone Solvate/StarCap 40 mg HGCs (Hard Gelatin Capsule)

Capsule formulation Vinorelbine monotartrate was premixed with approximately half of the dispensed StarCap 1500, passed through a screen and collected in an intermediate bulk container. The screen was flushed with the remaining StarCap 1500 and collected. The contents of the intermediate bulk container were blended until the contents were uniform. A hard gelatin size 2 was filled with vinorelbine monotartate and co-processed mixture of corn starch and pregelatinized starch. The capsule contained approximately 48 mg of vinorelbine monotartrate (corresponding to 40.00 mg of vinorelbine) and approximately 72.00 mg of StarCap 1500. Bulk characteristics were as follows: angle of response 24, bulk density 0.581 g/ml, tapped density 0.714 g/ml, Hausner ratio 1.229, LoD determination 3.79%. Bulk particle distribution data showed about 60% of particles to be of size of 0.08 mm, 18% of size 0.125 mm.

Dissolution of vinorelbine monotartrate HGC has been tested in 900 ml 0.1 N HCl at 75 rpm at 37° C. and compared with commercial batch of Navelbine Oral 30 mg SGC (soft gelatin capsule). The dissolution profile shows vinorelbine monotartrate HGC achieved a release of about 98% after 45 min while the vinorelbine SGC achieved a release of 97% after 45 min (Table 13).

TABLE 13
Comparison of dissolution kinetics of Navelbine Oral and
the hard gelatin capsules according to the present invention

| Time (min) | 0 | 10 | 20 | 30 | 45 |
|---|---|---|---|---|---|
| Amount of dissolved API (%) Navelbine Oral | 0 | 93 | 95 | 95 | 97 |
| Amount of dissolved API (%) hard gelatin capsules | 0 | 93 | 95 | 97 | 98 |

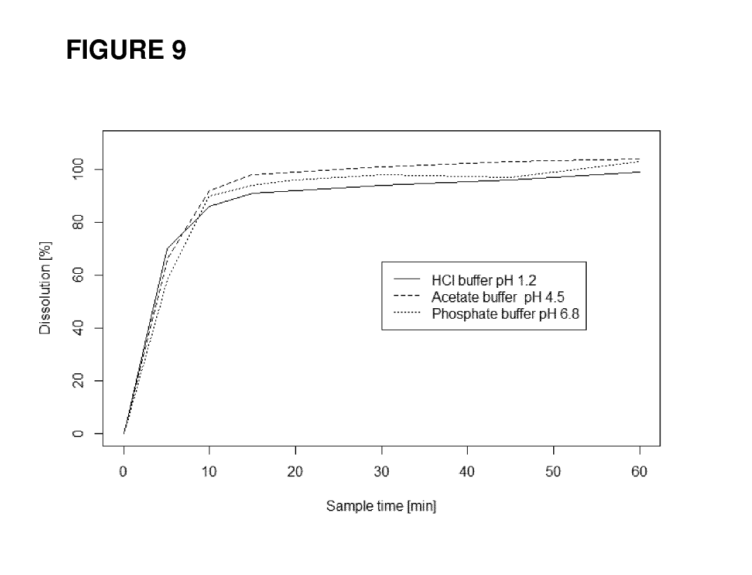

When dissolution profiles of 30 mg HGCs in three different dissolution media were compared, no significant difference has been identified. In all three media the HGCs dissolution complies with a general requirement NLT (not less than) 85% in NMT (not more than) 15 min (FIG. 9).

### Example 12. Preparation of Vinorelbine Monotartrate Hydrate/StarCap 30 mg HGCs

Capsule formulation Vinorelbine monotartrate hydrate was premixed with approximately half of the dispensed StarCap 1500, passed through a screen and collected in an intermediate bulk container. The screen was flushed with the remaining StarCap 1500 and collected. The contents of the intermediate bulk container were blended until the contents were uniform. A hard gelatin size 3 was filled with vinorelbine monotartate and co-processed mixture of corn starch and pre-gelatinized starch. The capsule contained approximately 36.00 mg of vinorelbine monotartrate (corresponding to 30.00 mg of vinorelbine base) and approximately 114.00 mg of StarCap 1500. Bulk characteristics were as follows: flowability 1.2-2 sec/100 g, angle of response 26, bulk density 0.51 g/ml, tapped density 0.66 g/ml, Hausner ratio 1.29, LoD determination 1.0%. Bulk particle distribution data showed about 20% of particles to be of size of 0.2 mm, 18% of size 0.31 mm and more than 50% of size of 0.5 mm.

Dissolution of vinorelbine monotartrate HGCs has been tested in 900 ml 0.1 N HCl at 75 rpm at 37° C. and compared with commercial batch of Navelbine Oral 30 mg SGC. The dissolution profile shows vinorelbine monotartrate HGC achieved a release of about 97% after 45 min while the vinorelbine SGC achieved a release of 98% after 45 min (Table 13).

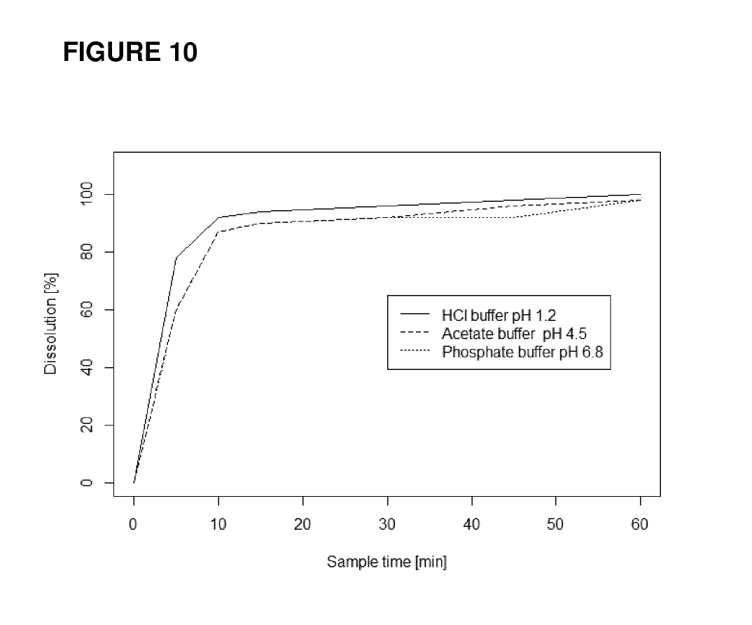

When dissolution profiles of 30 mg HGCs in three different dissolution media were compared, no significant difference has been identified. In all three media the HGCs dissolution complies with a general requirement NLT 85% in NMT 15 min (FIG. 10).

As evident from Table 14 and Table 15 analytical data showed the formulation complied with all studied requirements, including content uniformity, assay/purity, water disintegration.

TABLE 14
Content Uniformity (limits142.5 to 157.5 mg)

| | | Capsule filling weight |
|---|---|---|
| Sample | Assay [%]* | [mg] |
| 1 | 99.40 | 153.7 |
| 2 | 96.60 | 149.1 |
| 3 | 95.44 | 148.9 |
| 4 | 92.88 | 146.2 |
| 5 | 95.59 | 145.8 |
| 6 | 101.27 | 156.3 |
| 7 | 94.74 | 147.1 |
| 8 | 94.10 | 146.6 |
| 9 | 103.18 | 153.2 |
| 10 | 97.60 | 149.2 |
| mean | 97.08 | 149.6 |
| SD [%] | 3.29 | |
| RSD [%] | 3.39 | |
| AV | 9.3 | |

TABLE 15
Disintegration in Water

| Sample | 1 | 2 | 3 | 4 | 5 | 6 | Mean |
|---|---|---|---|---|---|---|---|
| Time | 1:51 | 2:57 | 2:07 | 1:47 | 2:03 | 1:43 | 2:04 |
| [min] | | (max) | | | | (min) | |

The results of the examples 11 and 12 showed crystalline vinorelbine monotartrate can be formulated with co-processed starch, providing simple and robust formulation, free from other excipients, ready for scale up.

### Example 13: Stability of Crystalline Vinorelbine Monotartrate Hydrate Containing Capsule Formulation

The stability of the vinorelbine monotartrate hydrate containing capsule formulation was determined at temperatures of 25° C. and of 40° C., respectively. The results obtained are summarized in Table 16. As evident from the table, the product remained stable during 6 months and at 25° C. and during 3 months at 40° C.

TABLE 16
Stability of capsule formulation at 25° C. and 40° C.

| Degradation products (%) | | |
|---|---|---|
| Photo degradation | Thermodegradation | |
| (3,6-epoxy | (4-deacetyl | Total |
| vinorelbine) | vinorelbine) | Limit |
| Limit NMT 0.30 | Limit NMT 0.15 | 0.70 |

storage conditions 25° C.

| | | | |
|---|---|---|---|
| initial | LT 0.05 | 0.06 | 0.06 |
| 15 days | LT 0.05 | 0.06 | 0.06 |
| 1 month | LT 0.05 | 0.06 | 0.07 |
| 2 months | LT 0.05 | 0.07 | 0.07 |
| 6 months | 0.05 | 0.07 | 0.12 |

storage conditions 40° C.

| | | | |
|---|---|---|---|
| initial | LT 0.05 | LT 0.05 | 0.06 |
| 15 days | LT 0.05 | LT 0.05 | 0.06 |
| 1 month | LT 0.05 | LT 0.05 | 0.08 |
| 2 months | LT 0.05 | LT 0.05 | 0.10 |
| 3 months | LT 0.05 | LT 0.05 | 0.10 |

TABLE 17 shows the results of a comparison of the stability of Navelbine Oral soft gelatin capsules and hard gelatin capsules of the present invention. Incubation was performed for six months at 25° C.±2° C. and 60%±2% relative humidity, as described above.

TABLE 17
Comparison of long-term stability at 25° C. of Navelbine
Oral soft gelatin capsules and hard gelatin capsules according to the
present invention

| | Navelbine Oral soft gelatin capsule | | |
|---|---|---|---|
| | Photodegradation | Thermodegradation | |
| | (%) | (%) | Total |
| | (3,6-epoxy | (4-deacetyl | (%) |
| | vinorelbine) | vinorelbine) | Limit |
| | Limit NMT 0.30 | Limit NMT 0.15 | 0.70 |
| initial | 0.06 | 0.05 | 0.20 |
| 6 months | 0.23 | 0.46 | 1.40 |
| | Hard gelatin capsule of the present invention | | |
| | Photo degradation | Thermodegradation | |
| | (3,6-epoxy | (4-deacetyl | Total |
| | vinorelbine) | vinorelbine) | Limit |
| | Limit NMT 0.30 | Limit NMT 0.15 | 0.70 |
| initial | LT 0.05 | 0.06 | 0.06 |
| 6 months | 0.05 | 0.07 | 0.12 |

The results obtained again reveal the virtual absence of degradation for the hard gelatin capsule formulation, whereas substantial degradation was observed for the established soft gelatin capsule.

### Example 14: Tablet Formulation

### 14.1 Direct Compression

Tablets employing as active ingredient vinorelbine monotartrate were produced by means of direct compression. Tablet cores containing approximately 36 mg of crystalline vinorelbine monotartrate (corresponding to 30 mg vinorelbine base), 85 mg microcrystalline cellulose (Avicel PH 102; Sigma-Aldrich, Munich, Germany), 10 mg StarCap 1500 (Colorcon, West Point, Pa., USA), 0.5 mg colloidal silica dioxide, and 1 mg magnesium stearate were prepared. The disintegration time of the tablets was determined to be approximately two minutes with a dissolution of >85% in 15 minutes. Furthermore, film coated tablets were prepared using the Opadry Film Coating System (Colorcon, West Point, Pa., USA).

### 14.2 Roller Compaction

Roller compaction dry granulation was used to prepare vinorelbine tablets. 36 mg of crystalline vinorelbine monotartrate (corresponding to 30 mg vinorelbine base), 85 mg microcrystalline cellulose (Avicel PH 102; Sigma-Aldrich, Munich, Germany), and 10 mg StarCap 1500 (Colorcon, West Point, Pa., USA) were mixed for 10 min. Intra-granular magnesium stearate was purified through a 250 μm sieve, added to a mixture and mixed for additional 5 min. The resulting mixture was compacted on a roller compactor. Colloidal silicon dioxide and few grams of granules were de-lumped by passing them through a 30 mesh screen. The mixture was added to the granules and blended for additional 5 min. Extra-granular magnesium stearate was also purified as described above, added and mixed for additional five minutes prior to compression. The disintegration time of the tablets was determined to be less than three minutes with a dissolution of >85% in 15 minutes.

The present invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modifications and variations of the inventions embodied therein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

1. A crystalline vinorelbine monotartrate.

2. The crystalline vinorelbine monotartrate according to claim 1, wherein the crystalline vinorelbine monotartrate is present as a solvate.

3. The crystalline vinorelbine monotartrate according to claim 2, wherein the crystalline vinorelbine monotartrate solvate contains an organic solvent and/or water.

4. The crystalline vinorelbine monotartrate according to claim 3, wherein the organic solvent is an alcohol, an ester, a ketone, an ether or a mixture thereof.

5. The crystalline vinorelbine monotartrate according to claim 4, wherein the organic solvent is acetone, diethyl ketone, ethyl acetate, isopropanol or a mixture thereof.

6. The crystalline vinorelbine monotartrate according to claim 2, wherein the crystalline vinorelbine monotartrate solvate contains less than 25% (w/w), preferably less than 20% (w/w), more preferably less than 15% (w/w), more preferably less than 10% (w/w), more preferably less than 5% (w/w), more preferably less than 2.5% (w/w) and most preferably less than 1.5% (w/w) organic solvent in the crystal structure of the crystalline vinorelbine monotartrate.

7. The crystalline vinorelbine monotartrate according to claim 5, wherein the organic solvent is acetone, isopropanol or a mixture thereof.

8. The crystalline vinorelbine monotartrate according to claim 2, wherein the crystalline vinorelbine monotartrate solvate is a hydrate.

9. The crystalline vinorelbine monotartrate according to claim 8, wherein the crystalline vinorelbine monotartrate hydrate contains 0.5-10% (w/w), preferably 3-7% (w/w), water and less than 2.5% (w/w), more preferably less than 1.5% (w/w), and most preferably less than 0.75% (w/w) organic solvent in the crystal structure of the crystalline vinorelbine monotartrate.

10. The crystalline vinorelbine monotartrate according to claim 8, wherein the crystalline vinorelbine monotartrate hydrate is characterized by a powder X-ray diffraction pattern comprising peaks at average diffraction angles (2Θ) of 7.9°, 9.5°, 10.3°, 10.8°, 13.4°, 13.6° and 14.5° (each ±0.2°).

11. The crystalline vinorelbine monotartrate according to claim 1, wherein the crystalline vinorelbine monotartrate is characterized by exhibiting a thermostability producing:
less than 0.1% degradation impurities of vinorelbine after 2 weeks at 25° C.±2° C., preferably less than 0.1% degradation impurities of vinorelbine after 1 month at 25° C.±2° C., more preferably less than 0.1% degradation impurities of vinorelbine after 3 months at 25° C.±2° C., more preferably less than 0.1% degradation impurities of vinorelbine after 6 months at 25° C.±2° C.,
more preferably less than 0.1% degradation impurities of vinorelbine after 2 weeks at 40° C.±2° C., more preferably less than 0.1% degradation impurities of vinorelbine after 1 month at 40° C.±2° C., more preferably less than 0.1% degradation impurities of vinorelbine after 3 months at 40° C.±2° C., and most preferably less than 0.1% degradation impurities of vinorelbine after 6 months at 40° C.±2° C.

12. The crystalline vinorelbine monotartrate according to claim 7, wherein the crystalline vinorelbine monotartrate is characterized by exhibiting a thermostability producing:
less than 0.1% degradation impurities of vinorelbine after 1 week at 60° C.±2° C., and/or less than 0.3% degradation impurities of vinorelbine after 8 weeks at 60° C.±2° C.

13. The crystalline vinorelbine monotartrate according to claim 1, wherein the crystalline vinorelbine monotartrate is characterized by exhibiting a photostability producing:
less than 0.1% 3,6-epoxy vinorelbine after an exposure of 15 minutes, less than 0.2% 3,6-epoxy vinorelbine after an exposure of 30 minutes, less than 0.3% 3,6-epoxy vinorelbine after an exposure of 60 minutes, and/or less than 0.5% 3,6-epoxy vinorelbine after an exposure of 120 minutes.

14. A method for producing the crystalline vinorelbine monotartrate according to claim 1, comprising the following steps:
(a) providing a solution of vinorelbine monotartrate in a liquid containing at least one organic solvent;
(b) drying the solution of vinorelbine monotartrate in a liquid containing at least one organic solvent until a dry residue is obtained;
(c) dissolving the dry residue in a liquid containing at least one organic solvent to obtain a mixture;
(d) maintaining the mixture under heating and stirring to obtain a solid precipitate;
(e) isolating the solid precipitate;
(f) drying the solid precipitate.

15. The method according to claim 14, wherein the method further comprises the step of:
(g) exposing the dried solid precipitate to water vapour.

16. The method according to claim 14, wherein the liquid containing at least one organic solvent in steps (a) and (b) is methylene chloride.

17. The method according to claim 14, wherein the liquid in step (c) is a mixture of a water-miscible organic solvent and water.

18. The method according to claim 14, wherein a crystalline vinorelbine monotartrate solvate is used as starting material.

19. A pharmaceutical composition comprising the crystalline vinorelbine monotartrate according to claim 1.

20. The pharmaceutical composition according to claim 19, wherein the pharmaceutical composition comprises the crystalline vinorelbine monotartrate and at least one pharmaceutically acceptable excipient, preferably the at least one pharmaceutically acceptable excipient is a co-processed excipient.

21. The pharmaceutical composition according to claim 19, wherein the pharmaceutical preparation is in an oral dosage form.

22. The pharmaceutical composition according to claim 21, wherein the oral dosage form is a solid dosage form, preferably selected from the group consisting of capsules, tablets, pills, dragees, granules, pellets, and powders.

23. The pharmaceutical composition according to claim 19, wherein the crystalline vinorelbine monotartrate in the pharmaceutical composition is characterized by exhibiting a thermostability producing:
less than 0.1% degradation impurities of vinorelbine after 2 weeks at 25° C.±2° C., preferably less than 0.1% degradation impurities of vinorelbine after 1 month at 25° C.±2° C., more preferably less than 0.1% degradation impurities of vinorelbine after 2 months at 25° C.±2° C., more preferably less than 0.1% degradation impurities of vinorelbine after 6 months at 25° C.±2° C.,
more preferably less than 0.1% degradation impurities of vinorelbine after 2 weeks at 40° C.±2° C., more preferably less than 0.1% degradation impurities of vinorelbine after 1 month at 40° C.±2° C., more preferably less than 0.1% degradation impurities of vinorelbine after 2 month at 40° C.±2° C. and most preferably less than 0.1% degradation impurities of vinorelbine after 3 months at 40° C.±2° C.

24. A method of preventing or treating cancer, comprising administering to a subject a pharmaceutical composition according to claim 19.

25. The method according to claim 24, wherein the cancer is non-small cell lung cancer and/or breast cancer.

26-27. (canceled)
